(19) United States
(12) Patent Application Publication
(10) Pub. No.: US 2023/0046628 A1
(43) Pub. Date: Feb. 16, 2023

(54) Macromolecular compositions for binding small molecules

(71) Applicants: Stichting voor de Technische Wetenschappen, Utrecht (NL); Universiteit Utrecht Holding B.V., Utrecht (NL); UMC Utrecht Holding B.V., Utrecht (NL)

(72) Inventors: Robert Smakman, Nigtevecht (NL); Wilhelmus Everhardus Hennink, Utrecht (NL); Karin Gerarda Frederika Gerritsen, Utrecht (NL); Jacobus Adrianus Wilhelmus Jong, Utrecht (NL); Cornelus Franciscus van Nostrum, Utrecht (NL)

(73) Assignees: Stichting voor de Technische Wetenschappen, Utrecht (NL); Universiteit Utrecht Holding B.V., Utrecht (NL); UMC Utrecht Holding B.V., Utrecht (NL)

(21) Appl. No.: 17/777,639

(22) PCT Filed: Nov. 20, 2020
(86) PCT No.: PCT/EP2020/082895

§ 371 Date: May 18, 2022

(30) Foreign Application Priority Data
Nov. 22, 2019 (EP) 19210947.8

(51) Int. Cl. B01J 20/26; C08F 212/08; C08F 8/10; C08F 8/20; C08F 8/06; C08F 212/14; A61M 1/16

(52) U.S. Cl. CPC B01J 20/261; C08F 212/08; C08F 8/10; C08F 8/20; C08F 8/06; B01J 20/264; B01J 20/265; C08F 212/22; A61M 1/1696; A61M 1/1654

(57) ABSTRACT

The present invention relates to a method for preparing a macromolecular composition comprising phenylglyoxaldehyde-derivatives. The invention also relates to the macromolecular compositions per se, and to methods of using the macromolecular compositions. The macromolecular compositions are useful for undergoing subsequent reactions with small molecules, for instance to remove such small molecules from a solution.

### FIELD OF THE INVENTION

The present invention relates to a method for preparing a macromolecular composition comprising phenylglyoxaldehyde-derivatives. The invention also relates to the macromolecular compositions per se, and to methods of using the macromolecular compositions. The macromolecular compositions are useful for undergoing subsequent reactions with small molecules, for instance to remove such small molecules from a solution.

### BACKGROUND ART

Patients with end stage kidney disease (ESKD) or severe acute kidney failure undergo dialysis (either hemodialysis, or HD, or peritoneal dialysis, or PD) to replace kidney function. Although lifesaving, conventional dialysis has major shortcomings. The treatment is time-consuming and removal of waste molecules and excess water is inadequate, contributing significantly to poor life quality, severe health problems and high mortality (15-20% per year). Treatment costs are very high.

In dialysis, patient fluids are generally dialysed against a dialysis fluid, which is then discarded. It is desirable to regenerate the dialysis fluid, to allow use of smaller volumes. In miniaturisation efforts, patient fluids are dialysed against a relatively small amount of dialysis fluid, referred to as dialysate. During this process waste solutes from the patient fluid move towards the dialysate by diffusion and/or convection, often through a membrane such as a semipermeable membrane. If waste solutes are later removed from a dialysate, it can be reused, which is referred to as regeneration of dialysate. Efficient regeneration of dialysate would reduce the need for large volumes of dialysis fluid, making dialysis more practically implemented, less resource-dependent, and reducing waste streams.

A miniature artificial kidney device will be a major breakthrough in renal replacement therapy. Worldwide the number of dialysis patients has been estimated at 3.4 million (see www.fresenius.com/media_library/Fresenius_Annual_Report_2018.pdf). Currently, approximately 89% of the dialysis patients use HD techniques, either in a center (>96%) or at home (<4%) (see the ERA-EDTA Registry Annual Report 2017). While in-center HD requires long frequent visits to the hospital (about 3 times per week, 4 h per session), home HD offers more flexibility and autonomy. However, home HD still requires bulky dialysis machines and a large supply of dialysis fluids (at least 20 L per treatment) or a bulky immobile water purification system. A user-friendly lightweight HD device that is independent of a fixed water supply or large supply of dialysis fluids will increase patients' mobility allowing them to stay active in social life and travel freely.

The large fluctuations in water balance and uremic toxin levels between dialysis treatments with standard thrice weekly HD could be attenuated with continuous or more frequent HD, which may improve patient outcome (Nesrallah 2012; Ting 2003; Susantitaphong 2012). A more liberal diet would be allowed. Significant cost reductions will be achieved through reduced need of dialysis personnel and related infrastructure, fewer medications and less hospitalizations due to reduced comorbidity.

PD is currently used by approximately 11% of the dialysis patients (see Fresenius 2018 annual report). Although PD, in contrast to HD, offers the opportunity to dialyze continuously, the technique has some major drawbacks: uremic toxin clearance is low (Evenpoel 2006), exchange procedures are time-consuming and technique failure rate is high (median technique survival is 3.7 years) due to high incidence of infection of the peritoneal membrane (peritonitis) and membrane failure (Perl 2012). The low dialysis efficacy is largely due to fast dissipation of the concentration gradient between plasma and peritoneal dialysate during a dwell, thereby limiting solute transport (Gotch 2001). A miniature PD device that continuously regenerates the dialysate, thereby maintaining the plasma-dialysate concentration gradient, would greatly enhance PD efficacy. This will allow reduction in the number of time consuming exchanges while still improving waste solute clearance. In addition, reducing the number of connections decreases the risk of contamination and will lower peritonitis rates (Piraino 2010; De Fijter, 1991). Continuous glucose infusion by the miniature PD device will reduce functional deterioration of the peritoneal membrane by avoiding very high toxic glucose concentrations as applied in conventional PD (Gotch 2001). By preventing the two major causes of technique failure in conventional PD (recurrent infection and functional loss of the peritoneal membrane) the miniature artificial kidney will significantly prolong technique survival.

A user-friendly wearable or portable dialysis device, providing dialysis outside the hospital, would thus represent a huge leap forward for dialysis patients and would significantly increase their quality of life. The device would allow continuous or more frequent dialysis which will improve removal of waste solutes and excess fluid, and hence patient health. A miniaturized design, independent of a fixed water supply, offers freedom and autonomy to the patient.

In recent years, small prototype dialysis devices have been constructed that adequately remove some organic waste solutes and waste ions. However, thus far no adequate strategy for removal of urea exists that allows miniaturization to truly wearable proportions, which is one of the main obstacles for successful realization of a miniature artificial kidney device. Urea is the waste solute with the highest daily production (primary waste product of nitrogen metabolism) and exerts toxic effects at high plasma concentrations. However, urea is difficult to bind and has low reactivity.

DE2305186A1/U.S. Pat. No. 3,933,753A discloses a macromolecular composition wherein a polystyrene-like scaffold is post-modified to comprise glyoxal moieties, reaching a conversion of 0.72 glyoxal moieties per monomer in the composition. This composition captured up to 1 mmol/g urea, and it was shown to be more suitable for the removal of aniline, which is not clinically relevant. This material was further developed as described in U.S. Pat. No. 4,012,317, but urea capture levels remained at comparable levels. WO2004078797A1 discloses similar ketoaldehyde materials, reaching a urea binding capacity of 1.5 mmol/g.

EP121275A1/U.S. Pat. No. 4,897,200A discloses a ninhydrin-type sorbent that is formed out of a polymerized styrene composition in a six-step synthetic sequence. A urea binding capacity of 1.2 mmol/g dry sorbent in 8 hours was shown at clinically relevant urea concentrations. However, for effective miniaturisation, a higher urea binding capacity is required. WO2019110557 discloses a ninhydrin-based material that has a urea-binding capacity of over 2 mmol/g.

U.S. Pat. No. 4,178,241A discloses a polystyrene-based material comprising para-thio, para-nitro, or para-amino moieties. Only for thio moieties, the binding of urea was again shown to be at about 1.5 mmol/g. Other functional groups performed less well. On the other hand, creatinine was shown to be bound at well over 90% of normal adult daily production for each functional group.

WO2017116515A1 discloses the use of electrically charged membranes to improve urea separation from a dialysis fluid, and suggests the use of electrooxidation of separated urea. A disadvantage of this method is that reactive oxygen species are generated as a byproduct.

WO2011102807A1 discloses epoxide-covered substrates. The epoxides can be used to recover solutes from a solution. They are also used to immobilise urease enzymes, which help dispose of urea. A disadvantage of urease enzymes is their sensitivity to environmental factors, their costly and laborious production, and the fact that ammonium is generated by their reaction, which in turn requires removal using cation exchangers comprising toxic materials such as zirconium phosphate. WO2016126596 also uses a very different substrate, viz. reduced graphene oxide. While a high urea binding capacity was shown, the captured urea represented less than 15% of the initial urea concentration.

To enable the development of improved artificial kidney devices, there is an ongoing need for easily prepared sorbents that bind higher amounts of urea without the risk of leaching components into a dialysate, and without generating harmful side products.

### SUMMARY OF THE INVENTION

The invention provides a method for producing a phenylglyoxaldehyde (PGA)-type sorbent, comprising the steps of i) providing monomers of general formula (I):

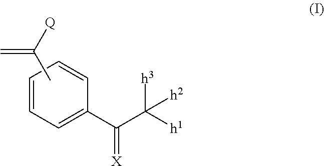

wherein Q is H or —CH₃; h¹, h², and h³ are each independently chosen from H, halogen, —OH, —O(C₁₋₆ hydrocarbon), —S(C₁₋₆ hydrocarbon), —NH(C₁₋₆ hydrocarbon), or —N(C₁₋₆ hydrocarbon)₂; or h¹ and h² together form ═O; or h¹ and h² together form -o¹—(C₁₋₄ hydrocarbon)-o²- wherein o¹ and o² are independently O, S, NH, or N(C₁₋₄ hydrocarbon); and X is O, S, or NH; followed by ii) polymerizing the provided monomers to obtain a polymer; and iii) converting polymerized monomers of general formula (I) that are not PGA-type monomers into PGA-type monomers. The monomers of general formula (I) preferably comprise monomers selected from 1-(4-ethenylphenyl)ethan-1-one, 1-(3-ethenylphenyl)ethan-1-one, 1-(4-isopropenylphenyl)ethan-1-one, 1-(3-isopropenylphenyl)ethan-1-one, 2-bromo-1-(4-ethenylphenyl)ethan-1-one, 2-bromo-1-(3-ethenylphenyl)ethan-1-one, 2-bromo-1-(4-isopropenylphenyl)ethan-1-one, 2-bromo-1-(3-isopropenylphenyl)ethan-1-one, 2-chloro-1-(4-ethenylphenyl)ethan-1-one, 2-chloro-1-(3-ethenylphenyl)ethan-1-one, 2-chloro-1-(4-isopropenylphenyl)ethan-1-one, 2-chloro-1-(3-isopropenylphenyl)ethan-1-one, 1-(4-ethenylphenyl)ethan-1,2-dione, 1-(3-ethenylphenyl)ethan-1,2-dione, 1-(4-isopropenylphenyl)ethan-1,2-dione, 1-(3-isopropenylphenyl)ethan-1,2-dione, 2,2-dihydroxy-1-(4-ethenylphenyl)ethan-1-one, 2,2-dihydroxy-1-(3-ethenylphenyl)ethan-1-one, 2,2-dihydroxy-1-(4-isopropenylphenyl)ethan-1-one, and 2,2-dihydroxy-1-(3-isopropenylphenyl)ethan-1-one.

Preferably, comonomers are provided along with the monomers of general formula (I). Preferably, the polymer is crosslinked after polymerization or during polymerization. Conversion in step iii) preferably comprises a step selected from: a) halogenation, preferably using halohydric acid; or b) oxidation, preferably using dimethyl sulfoxide (DMSO). Preferably, in step iii) more than 30%, 35%, 40%, 45%, 50%, 55%, 60%, 65%, 70%, 75%, 80%, 85%, 90%, 95%, or 100% of the monomers of general formula (I) are converted into PGA-type monomers. Preferably Q is H; and/or h¹ and h² are each independently chosen from H, halogen, —OH, and —O(C₁₋₄ hydrocarbon); or together form ═O; preferably h¹ and h² are both H, or are both —OH, or together form ═O; and/or h³ is H; and/or X is O.

The invention also provides a PGA-type sorbent obtainable by the method, wherein the sorbent has a urea binding capacity of more than 1.60 mmol urea per gram of sorbent. Preferably at least 30%, 35%, 40%, 45%, 50%, 55%, 60%, 65%, 70%, 75%, 80%, 85%, 90%, 95%, or 100% of the polymerized monomers is a PGA-type monomer. The invention also provides a composition comprising the PGA-type sorbent and a pharmaceutically acceptable excipient. The sorbent and the composition can be for use as a medicament, preferably for use in the treatment of a disease or condition associated with accumulation of urea.

The invention also provides a method for removing nucleophilic waste solutes from a fluid, comprising the steps of i) providing a fluid comprising nucleophilic waste solutes, and iia) contacting said fluid with a PGA-type sorbent according to the invention, or with a composition as defined above, or alternately iib) contacting said fluid with a dialysis fluid through a membrane, wherein the dialysis fluid is in contact with a PGA-type sorbent as defined in claim 9 or 10, or with a composition as defined in claim 11, and iii) optionally, recovering the fluid.

The invention further provides a cartridge for use in a dialysis device, comprising a PGA-type sorbent as defined above, or comprising a composition as defined above. Also provided is a dialysis device comprising a PGA-type sorbent as defined above, a composition as defined above, or a cartridge as defined above.

### DESCRIPTION OF EMBODIMENTS

The present invention seeks to provide an improved phenylglyoxaldehyde (PGA)-type sorbent with an increased capacity for nucleophilic waste solutes, of which urea is an important example. A sorbent is a material that binds target substances—in this case the sorbent binds nucleophiles such as urea. The inventors invented macromolecular dicarbonyl compounds with high urea binding capacity and fast binding kinetics, rendering the materials suitable for use as sorbents. The improved sorbent allows for the miniaturisation of sorbent cartridges and is thus an important step towards a miniature artificial kidney device.

The sorbent can advantageously be used in (hemo)dialysis for the removal of urea, wherein blood is led past a membrane such as a semipermeable membrane that separates it from a small amount of dialysis fluid. It can also be used in peritoneal dialysis for removal of urea from the peritoneal dialysate or for regeneration of the peritoneal dialysate, e.g. in continuous flow (or tidal flow) peritoneal dialysis with (continuous) regeneration of the dialysate. The sorbent then binds nucleophilic waste solutes such as urea, so that diffusion of these solutes over the semipermeable membrane is continued and does not slow down due to saturation.

Because effective urea removal is crucial for successful dialysate regeneration, an object of the present invention is to provide sorbents with high binding capacity, suitable for application in a miniature artificial kidney device, for example by preparing cartridges loaded with the sorbent. Sorbents of the invention have a binding capacity that is higher than that of known PGA-type sorbents. As demonstrated in example 4, sorbents according to the invention can bind at least 1.8 mmol urea per gram of sorbent, and can bind more. Sorbents prepared according to known methods (viz. that of WO2004078797A1) bind about 1.4 mmol/gram (WO2004078797 reports 1.5 mmol/gram). Another object of the invention is to provide a method for producing such an improved sorbent, preferably in a cost-effective matter by using low-cost reactants, thus allowing a reduction in the cost of healthcare. Another object of the invention is to provide a method wherein such sorbents, compositions, or cartridges are used to remove nucleophilic solutes such as urea or biologicals from a solution.

The inventors have surprisingly found that an improved PGA-type sorbent can be formed by polymerizing precursor monomers, and by subsequent conversion of these polymerized precursor monomers into PGA monomers. In the state of the art, PGA is formed based on styrene. The invention uses monomers that are structurally more close to PGA, such as vinylphenylethan-1-one (VPE, also known as 1-(4-ethenylphenyl)ethan-1-one and p-acylstyrene), vinylphenylethan-1,2-dione, or (hemi)acetals thereof. Surprisingly, the sorbents formed by the method of the invention have an improved capacity for binding urea, and thus allow improved methods for their use. An important improvement lies in the increased binding capacity of the produced sorbents.

The inventors surprisingly found that improved PGA-type sorbents can be obtained by using precursor monomers that resemble PGA more closely than styrene does. As part of the invention a family of suitable precursor monomers was found. Accordingly, in a first aspect, the invention provides a method for producing a PGA-type sorbent, comprising the steps of:
i) providing a monomer of general formula (I):

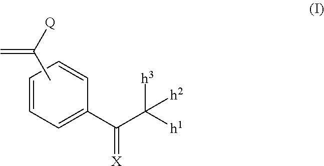

wherein:
Q is H or —CH₃;
h¹, h², and h³ are each independently chosen from H, halogen, —OH, —O(C₁₋₆ hydrocarbon), —S(C₁₋₆ hydrocarbon), —NH(C₁₋₆ hydrocarbon), or —N(C₁₋₆ hydrocarbon)₂; or h¹ and h² together form ═O; or h¹ and h² together form -o¹—(C₁₋₄ hydrocarbon)-o²- wherein o¹ and o² are independently O, S, NH, or N(C₁₋₄ hydrocarbon); and
X is O, S, or NH;
ii) polymerizing the provided monomers to obtain a polymer; and
iii) converting polymerized monomers of general formula (I) that are not PGA-type monomers into PGA-type monomers.

Such a method is hereinafter referred to as a production method according to the invention. A PGA-type sorbent obtainable by a production method according to the invention is hereinafter referred to as a PGA-type sorbent according to the invention.

PGA-Type Sorbent

A sorbent is a material that is designed to bind, absorb, or adsorb other substances. Sorbents for binding nucleophilic waste solutes are known in the art, and have already been described for use in hemodialysis devices (EP121275A1). In the context of this invention, a sorbent is a macromolecular composition that is a solid, a suspended solid, a colloidal suspension, an aggregate, a resin, or a polymer that can be dissolved or partially dissolved. It can bind nucleophilic waste solutes, after which the sorbent can be recovered from a mixture. The binding can be covalent, or non-covalent such as via electrostatic interactions or via hydrophobic interactions. Preferably the binding of a nucleophilic waste solute by a sorbent, particularly by a PGA-type sorbent according to the invention, is covalent.

A PGA-type sorbent is a sorbent that comprises PGA-type moieties. PGA is phenylglyoxaldehyde or 1-phenylethan-1,2-dione or phenyloxaldehyde. A PGA-type moiety is preferably a short aliphatic structure, preferably having only two carbon atoms, featuring two vicinal carbonyl groups (or a hydrate thereof), attached to an aromatic ring or aromatic ring-system, preferably attached to a phenyl moiety or a substituted phenyl moiety (for example substituted with a polymeric backbone). A glyoxaldehyde and its hydrate readily convert into one another, and it is to be understood that reference to PGA generally also entails reference to its hydrate. Generally, a hydrate of a glyoxaldehyde forms in non-dry environments, and the glyoxaldehyde can be dehydrated through heating; in aqueous environments both species generally coexist in an equilibrium. Preferred examples of PGA-type moieties are selected from the group consisting of orto-oxaldehydylphenyl, meta-oxaldehydylphenyl, and para-oxaldehydylphenyl, and their hydrates, wherein the phenyl ring can be optionally further substituted. In some embodiments the PGA-type moieties are selected from the group consisting of orto-oxaldehydylphenyl and meta-oxaldehydylphenyl. In other embodiments the PGA-type moieties are selected from the group consisting of orto-oxaldehydylphenyl and para-oxaldehydylphenyl. In some embodiments the PGA-type moieties are selected from the group consisting of meta-oxaldehydylphenyl and para-oxaldehydylphenyl. Most preferably the PGA-type moieties are para.

PGA-type sorbents according to the invention are suitable for binding nucleophilic waste solutes and do so at high capacity. These solutes react with the PGA-like moieties comprised in the sorbent. Accordingly, in a second aspect the invention provides a PGA type sorbent according to the invention, obtainable by a production method according to the invention, wherein the sorbent has a urea binding capacity of more than 1.60, preferably of more than 1.80, more preferably more than 2.00 mmol urea per gram of sorbent.

Urea is a small, highly polar molecule that, by virtue of its polarity and capability to participate in hydrogen bond formation, is highly soluble in water (>400 mg/ml) and in protic organic solvents such as methanol, ethanol, and glycerol. While the role of urea in biochemistry is essential, and it is an important molecule industrially, including as a source of nitrogen for fertilizer and as a polymer precursor, it is often important for urea to be removed from fluid solutions.

The production method according to the invention yields PGA-type sorbents that have a surprisingly high capacity for binding nucleophilic waste solutes such as urea. Urea generally reacts with the dehydrated PGA-type sorbent (see FIG. 1). Without wishing to be bound by theory, it is speculated that the more efficient conversion of precursor monomers into PGA-type monomers contributes to this increased binding capacity. In preferred embodiments of this aspect, the invention provides a PGA-type sorbent according to the invention, wherein the sorbent has a urea binding capacity of more than 1.51 mmol urea per gram of sorbent. In more preferred embodiments of this aspect, the invention provides a PGA-type sorbent according to the invention, wherein the sorbent has a urea binding capacity of more than 1.60 mmol urea per gram of sorbent. In further preferred embodiments of this aspect, the invention provides a PGA-type sorbent according to the invention, wherein the sorbent has a urea binding capacity of more than 1.51, 1.52, 1.53, 1.54, 1.55, 1.56, 1.57, 1.58, 1.59, 1.60, 1.61, 1.62, 1.63, 1.64, 1.65, 1.67, 1.68, 1.69, 1.70, 1.71, 1.72, 1.73, 1.74, 1.75, 1.76, 1.77, 1.78, 1.79, 1.80, 1.85, 1.90, 1.95, 2.00, 2.05, 2.10, 2.15, 2.20, 2.25, 2.30, 2.35, 2.40, 2.45, 2.50, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, or 4.9 mmol urea per gram of sorbent; more preferably the sorbent has a urea binding capacity of more than 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, or 4.9 mmol urea per gram of sorbent; even more preferably the sorbent has a urea binding capacity of more than 1.80, 1.90, 2.00, 2.10, 2.20, 2.25, 2.30, 2.35, 2.40, 2.45, 2.50, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, or 4.9 mmol urea per gram of sorbent; still more preferably the sorbent has a urea binding capacity of more than 2.00, still more preferably more than 2.20, even more preferably of more than 2.40, 2.45, 2.50, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, or 4.9 mmol urea per gram of sorbent, such as of more than 2.5 or of more than 2.6 mmol urea per gram of sorbent. Alternately, the sorbent has a urea binding capacity that is 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5 times as high as that of a reference sorbent, wherein the reference sorbent is preferably the sorbent as described in experiment 9 of WO2004/078797, which in experiment 16 of that publication is reported have the highest urea uptake of that publication (namely 90 mg urea per gram of sorbent). In this aspect the invention also provides a PGA-type sorbent according to the invention, wherein the sorbent has a urea binding capacity of more than 1.8, preferably 2 mmol urea per gram of sorbent or more preferably of more than 2.1 mmol urea per gram of sorbent, which is particularly suitable for miniaturisation of an artificial kidney device or of a (hemo)dialysis device.

In this context, the urea binding capacity is preferably the maximum urea binding capacity, which is preferably the capacity that can be determined after incubation of a sorbent with an excess of urea in a solution (such as about 30 mM) at about 70° C. for about 24 hours. The amount of bound urea can be determined by directly analysing the amount of urea bound to the sorbent, or by analysing the difference between the amount of urea present in the solution before and after exposure to the sorbent, or by regenerating the sorbent by dissociating the bound urea, and subsequent determination of the amount of released urea. Urea concentration can be determined by any method known in the art, such as by elemental analysis as described in WO2004078797. Alternately, the amount of ammonia released by an urease enzyme can be used to indirectly quantify urea concentrations. Alternately, a PAB reagent solution containing about 4% (w:v) of 4-(dimethylamino)benzaldehyde and 4% (v:v) sulphuric acid in absolute ethanol can be used for UV-VIS analysis (422 nm) of the urea reaction adduct using a previously prepared calibration curve, as described in WO2016126596. Various kits for determining urea concentration are commercially available, and contain instructions for use.

In PGA-type sorbents according to the invention preferably at least 5%, 10%, 15%, 20%, 25%, 30%, 35%, 40%, 45%, 50%, 55%, 60%, 65%, 70%, 75%, 80%, 85%, 90%, 95%, or 100% of the polymerized monomers is a PGA-type monomer. A preferred PGA-type sorbent according to the invention is a PGA-type sorbent wherein at least 30%, 35%, 40%, 45%, 50%, 55%, 60%, 65%, 70%, 75%, 80%, 85%, 90%, 95%, or 100% of the polymerized monomers is a PGA-type monomer, preferably at least 50%, more preferably at least 60%, even more preferably at least 70%, still more preferably at least 80%, and most preferably at least 90%. In further preferred PGA-type sorbents according to the invention, at least 50% and at most 90% of the polymerized monomers is a PGA-type monomer. In more preferred embodiments, 55% to 90% of the polymerized monomers is a PGA-type monomer. In even more preferred embodiments, 70% to 90% of the polymerized monomers is a PGA-type monomer. In most preferred embodiments, 70% to 80% of the polymerized monomers is a PGA-type monomer. The amount of PGA-type monomer is preferably reported as the amount of monomer of general formula (I) that was used during polymerization. Alternately, the amount of PGA-type monomer can be assessed using conventional techniques known in the art, such as solid state NMR or IR spectroscopy, as exemplified in the examples.

In a particular embodiment of this aspect the invention provides a PGA-type sorbents according to the invention wherein 100% of the polymerized monomers is a PGA-type monomer. Such a polymer is particularly suitable for use outside of resins, such as in a composition or a pharmaceutical composition for oral administration.

In preferred embodiments, the PGA-type sorbent according to the invention is obtainable by a production method according to the invention wherein the polymerization is suspension polymerisation. In preferred embodiments, the PGA-type sorbent according to the invention is obtainable by a production method according to the invention wherein at most 50%, preferably at most 35%, more preferably at most 25%, even more preferably at most 20%, most preferably at most 10% cross-linking monomer is used.

PGA-type sorbents according to the invention are obtainable by a production method according to the invention, and can thus comprise comonomers and crosslinks as described for the method of the invention.

Method of Production

As described, the first aspect of the invention provides a method comprising the steps of:
i) providing monomers of general formula (I):

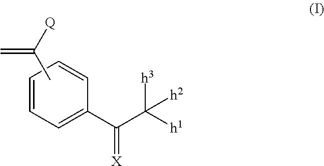

wherein:
Q is H or —CH₃;
h¹, h², and h³ are each independently chosen from H, halogen, —OH, —O(C₁₋₆ hydrocarbon), —S(C₁₋₆ hydrocarbon), —NH(C₁₋₆ hydrocarbon), or —N(C₁₋₆ hydrocarbon)₂; or h¹ and h² together form ═O; or h¹ and h² together form -o¹—(C₁₋₄ hydrocarbon)-o²- wherein o¹ and o² are independently O, S, NH, or N(C₁₋₄ hydrocarbon); and
X is O, S, or NH;
ii) polymerizing the provided monomers to obtain a polymer; and
iii) converting polymerized monomers of general formula (I) that are not PGA-type monomers into PGA-type monomers.

Step i)—Provision of a Monomer

In step i) a monomer is provided. The monomer can be synthesized using conventional techniques such as exemplified in the examples, or it can be procured from a commercial source. A monomer can already have a PGA-type moiety, or it can be converted into a monomer having a PGA-type monomer via subsequent reaction steps. Such a monomer that requires conversion is referred to herein as a precursor monomer. Preferably, a precursor monomer can be converted into a monomer comprising a PGA-type moiety in 1, 2, 3, or 4 reaction steps, more preferably in 1, 2, or 3 reaction steps, even more preferably in 1 or 2 reactions steps, most preferably in 1 reaction step. Because monomers of general formula (I) encompass both monomers with a PGA-type moiety and precursor monomers, it is preferred that a monomer of general formula (I) can be converted into a monomer comprising a PGA-type moiety in 0, 1, 2, 3, or 4 reaction steps, more preferably in 0, 1, 2, or 3 reaction steps, even more preferably in 0, 1, or 2 reaction steps, most preferably in 0 or 1 reaction steps. Reactions and reaction steps will be defined later herein, in the section detailing step iii).

The monomer features a handle for polymerization which is a vinyl (when Q is —H) or methylvinyl (when Q is —CH₃) moiety. Vinyl is also called ethenyl, and methylvinyl is also called isopropenyl, which should be interpreted as propenyl linked at its central carbon atom. This moiety comprising Q is linked to the ring that will be, after the conversion of step iii), the aromatic part of the PGA-type moiety. It can be in an orto, meta, or para position of the PGA-type moiety. Preferred monomers are of general formula (I-p) or (I-m), of which (I-p) is more preferred. When a less hydrophobic or more flexible PGA-type sorbent according to the invention is desired, it is preferred to use a monomer of general formula (I) wherein Q is —H, because this leads to a more flexible and less aliphatically bulky backbone of the resultant polymer.

The monomer further features a —C(═X)—C(h¹)(h²)(h³) moiety, which allows the monomer to be readily and efficiently converted into a PGA-type moiety in step iii), or which already forms a PGA-type moiety. A skilled person understands the valency of atoms, and understands that monomers of general formula (I) are to comply with such valency.

X is O, S, or NH; accordingly, X forms a ketone, a thione (thioketone), or an imine (ketamine). In preferred embodiments, X is S or NH. In other preferred embodiments, X is S or O. In other preferred embodiments, X is O or NH. In other preferred embodiments, X is S. In other preferred embodiments, X is NH. Most preferably, X is O.

h¹, h², and h³ form the remainder of the PGA-type monomer or precursor monomer of general formula (I). They are each independently chosen from H, halogen, —OH, —O(C₁₋₆ hydrocarbon), —S(C₁₋₆ hydrocarbon), —NH(C₁₋₆ hydrocarbon), or —N(C₁₋₆ hydrocarbon)₂; or h¹ and h² together form ═O; or h¹ and h² together form -o¹—(C₁₋₄ hydrocarbon)-o²- wherein o¹ and o² are independently O, S, NH, or N(C₁₋₄ hydrocarbon). This either forms a PGA-type monomer (for instance when h¹ and h² together form ═O and when h³ is H while X is O), or forms a precursor that can be readily converted into a PGA-type monomer as later described herein.

When each of h¹, h², and h³ form H, they form a —CH₃ moiety together with the carbon atom to which they are attached. This can be advantageous when the carbon atom is to be oxidized to form a PGA-type moiety. In preferred embodiments, each of each of h¹, h², and h³ form the same type of moiety, preferably H or halogen, more preferably H.

When h¹ and h² are both —OH and h³ is H, the result is a PGA-type moiety that is a hydrate, also known as a geminal-diol of a glyoxaldehyde. Such a group can be advantageous when the hydrate is to be converted into a PGA-type moiety via dehydration. In preferred embodiments, h¹ and h² are the same moiety or together form a single moiety. In other preferred embodiments, h¹ and h² together form a single moiety. For either of these, h³ is preferably H or halogen, more preferably H. For either of these, h¹ and h² are preferably-OH or halogen when not together forming a single moiety, more preferably —OH. In other preferred embodiment, h¹ and h² do not together form a single moiety.

When any of h¹, h², and h³ form —O(C₁₋₆ hydrocarbon), —S(C₁₋₆ hydrocarbon), —NH(C₁₋₆ hydrocarbon), or —N(C₁₋₆ hydrocarbon)₂, the resulting moieties can be hydrolyzed to yield —OH, or in the case of germinal moieties to yield ═O. —O(C₁₋₆ hydrocarbon) can contribute to formation of an acetal or hemiacetal. —S(C₁₋₆ hydrocarbon) can contribute to formation of a thioketal or a hemithioketal, and both —NH(C₁₋₆ hydrocarbon) and —N(C₁₋₆ hydrocarbon)₂ can contribute to formation of an aminal or hemiaminal. Accordingly, when at least one of h¹, h², and h³ form —O(C₁₋₆ hydrocarbon), —S(C₁₋₆ hydrocarbon), —NH(C₁₋₆ hydrocarbon), or —N(C₁₋₆ hydrocarbon)₂, it is preferred that at least one other of h¹, h², and h³ form —O(C₁₋₆ hydrocarbon), —S(C₁₋₆ hydrocarbon), —NH(C₁₋₆ hydrocarbon), —N(C₁₋₆ hydrocarbon)₂, or —OH, preferably —OH. In preferred embodiments, when at least one of h¹, h², and h³ form —O(C₁₋₆ hydrocarbon), —S(C₁₋₆ hydrocarbon), —NH(C₁₋₆ hydrocarbon), or —N(C₁₋₆ hydrocarbon)₂, a second of h¹, h², and h³ also forms —O(C₁₋₆ hydrocarbon), —S(C₁₋₆ hydrocarbon), —NH(C₁₋₆ hydrocarbon), or —N(C₁₋₆ hydrocarbon)₂.

As used herein a C₁₋₆ hydrocarbon is a hydrocarbon that has from 1 to 6 carbon atoms. As will be clear from context, it can be a single radical such as a methyl moiety, or a biradical forming a bridge between two moieties as indicated. It can be linear or branched, it can be saturated or unsaturated, and it can be optionally interrupted or substituted by heteroatoms such as O, N, or S, or substituted by methyl or phenyl. Preferred embodiments of C₁₋₆ hydrocarbon are C₁₋₆ alkyl, C₂₋₆ alkenyl, C₂₋₆ alkynyl, C₃₋₆ cycloalkyl, C₃₋₆ heterocycloalkyl, C₆ aryl, and C₅₋₆ heteroaryl. More preferred embodiments of C₁₋₆ hydrocarbon are C₁₋₆ alkyl, C₂₋₆ alkenyl, C₂₋₆ alkynyl, C₃₋₆ cycloalkyl, and C₆ aryl. In preferred embodiments C₁₋₆ denotes the number of carbon atoms in the longest internal chain of the hydrocarbon. In other preferred embodiments it denotes the total number of carbon atoms in the hydrocarbon. In preferred embodiments, a C₁₋₆ hydrocarbon is C₁₋₄, more preferably C₁₋₃, more preferably C₁₋₂. A skilled person will be able to select suitable hydrocarbons. Examples of suitable C₁₋₆ hydrocarbons are methyl, ethyl, n-propyl, isopropyl, butyl (including n-butyl, sec-butyl, tert-butyl, and isobutyl), pentyl, cyclopentyl, imidazolyl, phenyl, furyl, tetrahydrofuryl, and cyclohexyl. When two instances of C₁₋₆ hydrocarbon are attached to the same atom, they can together with said atom form a cyclic structure, preferably a 3-8 membered cyclic structure. Instances of C₁₋₄ hydrocarbon are preferably C₁₋₃ or C₁₋₂, or alternately preferably C₂₋₃, most preferably C₂. A hydrocarbon that connects two moieties (i.e. that is not terminal) preferably is at least a C₂ hydrocarbon such as —CH₂CH₂—. For -o¹—(C₁₋₄ hydrocarbon)-o²- a preferred embodiment is -o¹—C(phenyl)-o²-.

The above is reflected in the fact that h¹ and h² can also together form -o¹—(C₁₋₄ hydrocarbon)-o²- wherein o¹ and o² are independently O, S, NH, or N(C₁₋₄ hydrocarbon). This allows the presence of for example diol protecting groups for precursors of PGA-type monomers. As an example, when both o¹ and o² are O, and the connecting hydrocarbon is —CH₂CH₂—, h¹ and h² effectively form an acetal-protected PGA-type monomer. In preferred embodiments, o¹ and o² are the same. In preferred embodiments, o¹ and o² are selected from O, S, or NH. In more preferred embodiments, o¹ and o² are selected from O or S, most preferably o¹ and o² are O. A C₁₋₄ hydrocarbon that connects o¹ and o² is preferably a C₁₋₃ moiety, more preferably a C₂ moiety, most preferably it is —CH₂CH₂—. When present in N(C₁₋₄ hydrocarbon), it is preferably methyl, ethyl, or (iso)propyl, more preferably methyl or ethyl, most preferably methyl.

In preferred embodiments, h¹ and h² together form -o¹—(C₁₋₄ hydrocarbon)-o²- wherein o¹ and o² are independently O, S, NH, or N(C₁₋₄ hydrocarbon). More preferably h¹ and h² together form -o¹—(C₁₋₄ hydrocarbon)-o²- wherein o¹ and o² are independently O, S, or NH. Even more preferably h¹ and h² together form —O—(C₁₋₄ hydrocarbon)-O—. Still more preferably h¹ and h² together form -o¹—(C₂₋₄ hydrocarbon)-o²- wherein o¹ and o² are independently O, S, or NH. Even more preferably h¹ and h² together form —O—(C₂₋₃ hydrocarbon)-O—.

When none of h¹, h², and h³ together form a single moiety as described above, the following are preferred embodiments: h¹, h², and h³ are each independently chosen from halogen, —OH, —O(C₁₋₆ hydrocarbon), —S(C₁₋₆ hydrocarbon), —NH(C₁₋₆ hydrocarbon), or —N(C₁₋₆ hydrocarbon)₂; or from H, —OH, —O(C₁₋₆ hydrocarbon), —S(C₁₋₆ hydrocarbon), —NH(C₁₋₆ hydrocarbon), or —N(C₁₋₆ hydrocarbon)₂; or from H, halogen, —O(C₁₋₆ hydrocarbon), —S(C₁₋₆ hydrocarbon), —NH(C₁₋₆ hydrocarbon), or —N(C₁₋₆ hydrocarbon)₂; or from H, halogen, —OH, —S(C₁₋₆ hydrocarbon), —NH(C₁₋₆ hydrocarbon), or —N(C₁₋₆ hydrocarbon)₂; or from H, halogen, —OH, —O(C₁₋₆ hydrocarbon), —NH(C₁₋₆ hydrocarbon), or —N(C₁₋₆ hydrocarbon)₂; or from H, halogen, —OH, —O(C₁₋₆ hydrocarbon), or —S(C₁₋₆ hydrocarbon); or from H, halogen, or —OH; or from H, halogen, —OH, or —O(C₁₋₆ hydrocarbon); or from H or halogen; or from H or —OH; or from —OH or halogen.

Halogen in monomers of general formula (I) is preferably fluorine, chlorine, bromine, or iodine, more preferably chlorine, bromine, or iodine, even more preferably bromine or iodine, most preferably bromine.

In preferred monomers of general formula (I),
Q is H; and/or
h¹ and h² are each independently chosen from H, halogen, —OH, and —O(C₁₋₄ hydrocarbon); or together form ═O; preferably h¹ and h² are both H, or are both —OH, or together form ═O; and/or
h³ is H; and/or
X is O.

In other preferred monomers of general formula (I), Q is H; and h¹ and h² are each independently chosen from H, halogen, —OH, and —O(C₁₋₄ hydrocarbon); or together form ═O; preferably h¹ and h² are both H, or are both —OH, or together form ═O; and h³ is H; and X is O. In other preferred monomers of general formula (I), Q is H; and h¹ and h² are both H, or are both —OH, or together form ═O; and/or h³ is H; and/or X is O.

Monomers of general formula (I) can be of general formula (Io), of general formula (Ih), of general formula (I-Ac), of general formula (I-PGA), or of general formula (I-PGAH) as shown below:

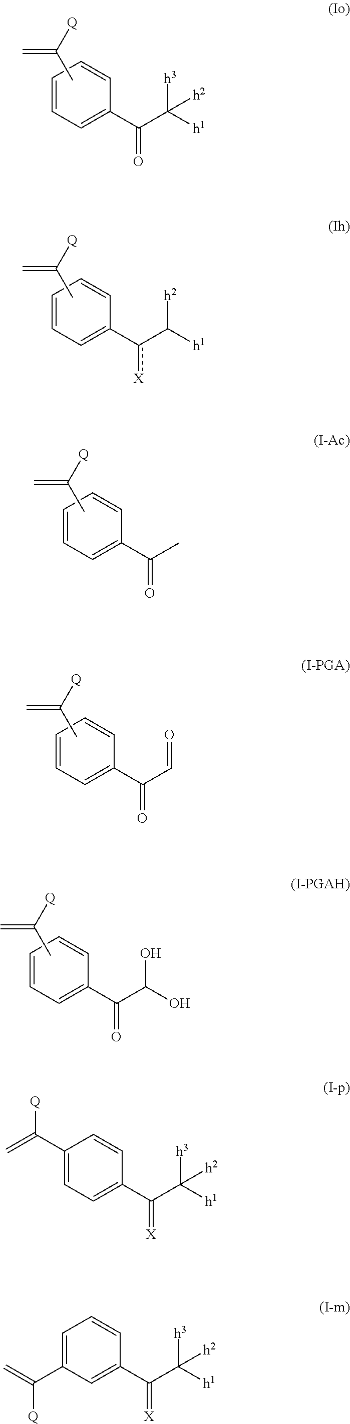

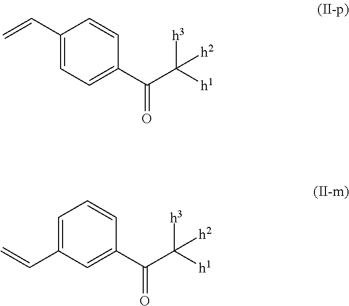

In preferred embodiments, a monomer of general formula (I) is of general formula (Io), wherein Q is —H or —CH₃ and wherein h¹ and h² and h³ are as defined above. A monomer of general formula (Io) is referred to herein as a ketone-type monomer.

In preferred embodiments, a monomer of general formula (I) is of general formula (Ih), wherein Q is —H or —CH₃ and wherein h¹ and h² are as defined above but not H, preferably wherein h¹ and h² together form a single moiety or are both the same but not H, more preferably together form a single moiety or are both halogen or —OH, most preferably both —OH. A monomer of general formula (Ih) is referred to herein as a ketal-type monomer because these monomers are particularly useful as acetals of PGA-type moieties.

In preferred embodiments, a monomer of general formula (I) is of general formula (I-Ac), wherein Q is —H or —CH₃. A monomer of general formula (I-Ac) is referred to herein as a VPE-type monomer because these monomers are particularly useful as vinylphenylethan-1-one analogues.

In preferred embodiments, a monomer of general formula (I) is of general formula (I-PGA) or (I-PGAH), preferably (I-PGA), wherein Q is —H or —CH₃. These monomers are referred to herein as a PGA-type monomers. In other preferred embodiments, the monomer is of general formula (II-p) or (II-m), of which (II-p) is preferred.

Preferred monomers of general formula (I) are shown below, with names shown below the structures.

| | | | |
|---|---|---|---|
| 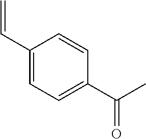 | 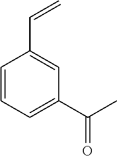 | 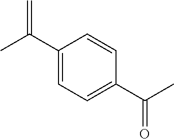 | 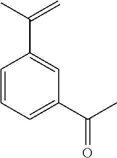 |
| 1-(4- | 1-(3- | 1-(4- | 1-(3- |
| ethenylphenyl)ethan- | ethenylphenyl)ethan- | isopropenylphenyl)ethan- | isopropenylphenyl)ethan- |
| 1-one | 1-one | 1-one | 1-one |
| 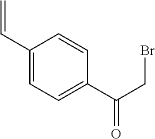 | 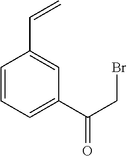 | 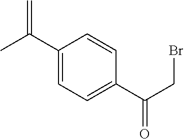 | 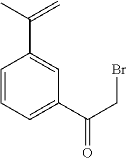 |
| 2-bromo-1-(4- | 2-bromo-1-(3- | 2-bromo-1-(4- | 2-bromo-1-(3- |
| ethenylphenyl)ethan- | ethenylphenyl)ethan- | isopropenylphenyl)ethan- | isopropenylphenyl)ethan- |
| 1-one | 1-one | 1-one | 1-one |
| 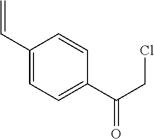 | 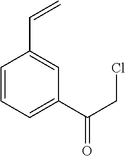 | 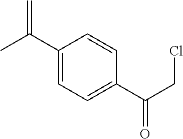 | 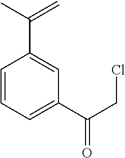 |
| 2-chloro-1-(4- | 2-chloro-1-(3- | 2-chloro-1-(4- | 2-chloro-1-(3- |
| ethenylphenyl)ethan- | ethenylphenyl)ethan- | isopropenylphenyl)ethan- | isopropenylphenyl)ethan- |
| 1-one | 1-one | 1-one | 1-one |
| 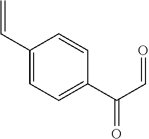 | 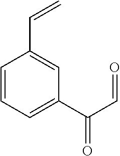 | 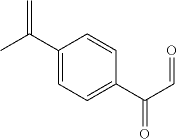 | 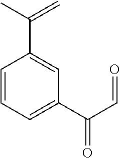 |
| 1-(4- | 1-(3- | 1-(4- | 1-(3- |
| ethenylphenyl)ethan- | ethenylphenyl)ethan- | isopropenylphenyl)ethan- | isopropenylphenyl)ethan- |
| 1,2-dione | 1,2-dione | 1,2-dione | 1,2-dione |
| 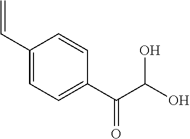 | 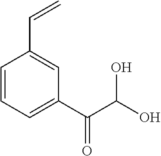 | 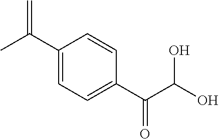 | 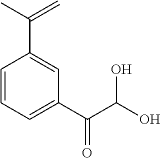 |
| 2,2-dihydroxy-1-(4- | 2,2-dihydroxy-1-(3- | 2,2-dihydroxy-1-(4- | 2,2-dihydroxy-1-(3- |
| ethenylphenyl)ethan- | ethenylphenyl)ethan- | isopropenylphenyl)ethan- | isopropenylphenyl)ethan- |
| 1-one | 1-one | 1-one | 1-one |
| 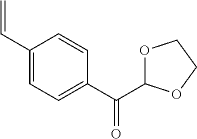 | 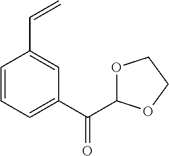 | 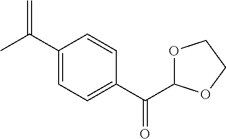 [SMILES: C=C(C)c1ccc(C(=O)C2OCCO2)cc1] | 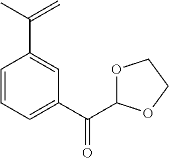 [SMILES: C=C(C)c1cccc(C(=O)C2OCCO2)c1] |
| (4-ethenylphenyl) | (3-ethenylphenyl) | (4-isopropenylphenyl) | (3-isopropenylphenyl) |
| (2-dioxolanyl) | (2-dioxolanyl) | (2-dioxolanyl) | (2-dioxolanyl) |
| methanone | methanone | methanone | methanone |

In preferred embodiments of this aspect, the monomers of general formula (I) comprise at least one monomer selected from 1-(4-ethenylphenyl)ethan-1-one, 1-(3-ethenylphenyl)ethan-1-one, 1-(4-isopropenylphenyl)ethan-1-one, 1-(3-isopropenylphenyl)ethan-1-one,
2-bromo-1-(4-ethenylphenyl)ethan-1-one, 2-bromo-1-(3-ethenylphenyl)ethan-1-one, 2-bromo-1-(4-isopropenylphenyl)ethan-1-one, 2-bromo-1-(3-isopropenylphenyl)ethan-1-one,
2-chloro-1-(4-ethenylphenyl)ethan-1-one, 2-chloro-1-(3-ethenylphenyl)ethan-1-one, 2-chloro-1-(4-isopropenylphenyl)ethan-1-one, 2-chloro-1-(3-isopropenylphenyl)ethan-1-one,
1-(4-ethenylphenyl)ethan-1,2-dione, 1-(3-ethenylphenyl)ethan-1,2-dione, 1-(4-isopropenylphenyl)ethan-1,2-dione, 1-(3-isopropenylphenyl)ethan-1,2-dione,
2,2-dihydroxy-1-(4-ethenylphenyl)ethan-1-one, 2,2-dihydroxy-1-(3-ethenylphenyl)ethan-1-one, 2,2-dihydroxy-1-(4-isopropenylphenyl)ethan-1-one, and 2,2-dihydroxy-1-(3-isopropenylphenyl)ethan-1-one.

In further preferred embodiments, the invention provides the production method according to the invention, wherein at least one monomer of general formula (I) is comprise at least 1-(4-ethenylphenyl)ethan-1-one, 1-(3-ethenylphenyl)ethan-1-one, 2-bromo-1-(4-ethenylphenyl)ethan-1-one, or 2-bromo-1-(3-ethenylphenyl)ethan-1-one, optionally selected from isopropenyl analogues thereof.

In more preferred embodiments, the invention provides the production method according to the invention, wherein the monomer of general formula (I) is 1-(4-ethenylphenyl)ethan-1-one or 2-bromo-1-(4-ethenylphenyl)ethan-1-one, or optionally selected from isopropenyl analogies thereof.

In further preferred embodiments, the invention provides the production method according to the invention, wherein the monomer of general formula (I) is 1-(4-ethenylphenyl)ethan-1-one, or the isopropenyl analogue thereof.

It is particularly envisaged that provision of a monomer of general formula (I) may entail the provision of a mixture of monomers of general formula (I). Preferably, the compounds present in such a mixture differ only in their attachment position of the moiety comprising Q. As such:

when any one of 1-(4-ethenylphenyl)ethan-1-one or 1-(3-ethenylphenyl)ethan-1-one is provided, preferably a mixture of each of these monomers can also be provided;

when any one of 1-(4-isopropenylphenyl)ethan-1-one, 1-(3-isopropenylphenyl)ethan-1-one is provided, preferably a mixture of each of these monomers can also be provided;

in analogy, the same holds for the pairs of 2-bromo-1-(4-ethenylphenyl)ethan-1-one and 2-bromo-1-(3-ethenylphenyl)ethan-1-one; for 2-bromo-1-(4-isopropenylphenyl)ethan-1-one and 2-bromo-1-(3-isopropenylphenyl)ethan-1-one; for 2-chloro-1-(4-ethenylphenyl)ethan-1-one and 2-chloro-1-(3-ethenylphenyl)ethan-1-one; for 2-chloro-1-(4-isopropenylphenyl)ethan-1-one and 2-chloro-1-(3-isopropenylphenyl)ethan-1-one; for 1-(4-ethenylphenyl)ethan-1,2-dione and 1-(3-ethenylphenyl)ethan-1,2-dione; for 1-(4-isopropenylphenyl)ethan-1,2-dione and 1-(3-isopropenylphenyl)ethan-1,2-dione; for 2,2-dihydroxy-1-(4-ethenylphenyl)ethan-1-one and 2,2-dihydroxy-1-(3-ethenylphenyl)ethan-1-one; and for 2,2-dihydroxy-1-(4-isopropenylphenyl)ethan-1-one and 2,2-dihydroxy-1-(3-isopropenylphenyl)ethan-1-one.

The same is applicable for isopropenyl analogues of any of the above. Due to the synthetic accessibility of the various monomers, it is unlikely that isopropenyl and ethenyl analogues would be provided as a mixture, as these require different reactants.

Step ii)—Polymerization

In step ii), the monomers that were provided in step i) are polymerized to obtain a polymer. A polymer is a substance composed of macromolecules, wherein a macromolecule is a molecule of high relative molecular mass, the structure of which essentially comprises the multiple repetition of units derived, actually or conceptually, from molecules of low relative molecular mass. A polymer can be a single chain composed of one type of monomer, and a polymer can also be a large crosslinked network composed of many different monomers. As is known in the art, polymerization is the process of forming a polymer out of monomers.

Polymerization takes place via the moiety comprising Q, and can be via any method known in the art (see for instance S. M. Ashraf “A Laboratory Manual of Polymers” I. K. International Pvt Ltd, 8 Dec. 2008). Suitable methods for polymerizing vinyl or methylvinyl type monomers such as those of general formula (I) are radical polymerization, bond-insertion polymerization, and ionic polymerization. Examples of ionic polymerization are anionic polymerization and cationic polymerization. An example of bond-insertion polymerization is Ziegler-Natta polymerization. Examples of radical polymerization are free radical polymerization, atom transfer radical polymerization (ATRP), and radical addition fragmentation chain transfer polymerization (RAFT). Suspension polymerization using immiscible solvents is a preferred method because it can lead to a granulated sorbent. A skilled person knows how to perform such polymerizations.

Solvents can also be present as inert solvents; these are preferably dissolving inert solvents. Depending on the context an inert solvent can also be a mixture of inert solvents. The inert solvent can influence the porosity of the produced sorbent; accordingly the inert solvent can also be referred to as a porogen. A more polar inert solvent can lead to an increased porosity; a less polar inert solvent can lead to a more dense sorbent. A more polar inert solvent can lead to decreased mechanical stability of the resulting sorbent; a less polar inert solvent can lead to more mechanical stability in the resulting sorbent. Accordingly, the inert solvent preferably has a polarity that combines desirable porosity with desirable mechanical stability.

Suitable inert solvents are known in the art; examples are pentane, hexane, heptane, cyclohexane, benzene, toluene, xylene, nitrobenzene, nitromethane, furan, tetrahydrofuran, 1,4-dioxane, isoparaffin aliphatic hydrocarbons such as ShellSolTD (CAS 64761-65-7; Shell Chemicals product code for Europe: Q7411) or ShellSolT (CAS 64761-65-7; Shell Chemicals product code for Europe: Q7412), or mixtures thereof. Preferred inert solvents are pentane, hexane, heptane, cyclohexane, benzene, toluene, xylene, nitrobenzene, nitromethane, furan, tetrahydrofuran, 1,4-dioxane, ShellSolTD, ShellSolT, or mixtures thereof. More preferred inert solvents are pentane, hexane, heptane, cyclohexane, benzene, toluene, xylene, nitrobenzene, nitromethane, ShellSolTD, ShellSolT, or mixtures thereof. Even more preferred inert solvents are heptane, toluene, nitrobenzene, ShellSolTD, optionally nitromethane, or mixtures thereof; toluene is most preferred.

Mixtures of inert solvents are convenient when a specific polarity is desired, for example when more porosity is desired, an inert solvent with higher polarity should be selected. Examples of suitable mixtures of inert solvents are toluene:nitromethane, toluene:nitrobenzene; toluene:heptane; and toluene:ShellSolTD. Preferred mixtures of inert solvents are toluene:nitrobenzene (1:1); toluene:heptane (1:4); toluene:heptane (1:1); and toluene:ShellSolTD (1:4). More preferred mixtures of inert solvents are toluene:nitrobenzene (1:1); toluene:heptane (1:4); and toluene:heptane (1:1).

In preferred embodiments the inert solvent or mixture of inert solvents is not more polar than nitrobenzene and/or not less polar than heptane. In more preferred embodiments the inert solvent or mixture of inert solvents is not more polar than nitrobenzene and not less polar than heptane. In even more preferred embodiments the inert solvent or mixture of inert solvents is not more polar than nitrobenzene and/or not less polar than heptane:toluene (4:1). In most preferred embodiments the inert solvent or mixture of inert solvents is not more polar than nitrobenzene and/or not less polar than heptane:toluene (4:1). Polarity preferably refers to the average polarity of the inert solvent or of the average polarity of the mixture of inert solvents; it can be determined using any method known in the art, for example as described in Katritzky et al., Chem. Rev. (2004) DOI: 10.1021/cr020750m.

For productions methods according to the invention, it is preferred that step ii) entails polymerizing the provided monomer to obtain a polymer using radical polymerization, more preferably free radical polymerization. This is because the process of free radical polymerization is readily implemented and does not require complex setups or conditions. Suitable initiators for free radical polymerization are azobisisobutyronitrile (AIBN), benzoyl peroxide, benzoyl peroxide blend with dicyclohexyl phthalate, 1,1′-azobis(cyclohexanecarbonitrile) (ABCN), di-tert-butyl peroxide, acetone peroxide, methyl ethyl ketone peroxide, and peroxydisulfate salts such as sodium persulfate or potassium persulfate or ammonium persulfate. For aqueous systems, AIBN and/or peroxydisulfate salts, particularly potassium persulfate, are preferred. For suspension polymerization preferred initiators are benzoyl peroxide and/or benzoyl peroxide blend with dicyclohexyl phthalate.

During polymerization, a comonomer that is not of general formula (I) can also be present. Comonomers are further monomers that undergo the polymerization of step ii) in the same mixture as the monomers provided in step i), and comonomers become covalently incorporated in the resulting polymer. Such a resulting polymer is often referred to as a copolymer, but for sake of clarity this document will only refer to polymers as such, where the context will make it clear whether a copolymer could also be referenced. In the context of this invention, two classes of comonomers are particularly relevant: hydrophilic comonomers and crosslinking comonomers.

In preferred embodiments, the invention provides a production method according to the invention, wherein a comonomer is provided along with the monomer of general formula (I), wherein the comonomer is preferably selected from the group consisting of styrene, isopropenylbenzene, divinylbenzene, vinylbenzenesulfonic acid, acrylic acid, methacrylic acid, methyl acrylate, methyl methacrylate, ethyl acrylate, ethyl methacrylate, acrylonitrile, 2-hydroxyethyl 2-methylprop-2-enoate (HEMA), 2-hydroxypropyl 2-methylprop-2-enotate, 2-hydroxyethyl prop-2-enoate, 2-hydroxypropyl prop-2-enotate, N-(2-hydroxyethyl)methacrylamide, N-(2-hydroxypropyl)methacrylamide (HPMA), N-(2-hydroxyethyl)acrylamide, N-(2-hydroxypropyl)acrylamide, a telechelic N,N′-alkylenebisacrylamide such as N,N-methylenebisacrylamide (NMAA), N-isopropylacrylamide (NIPAm), divinyl sulfone, butadiene, methacrylonitrile, vinylsulfonamide, N-alkyl vinylsulfonamide such as N-methyl vinylsulfonamide, and N,N-dialkyl vinylsulfonamide such as N,N-dimethyl vinylsulfonamide. In more preferred embodiments, the invention provides a production method according to the invention, wherein a comonomer is provided along with the monomer of general formula (I), wherein the comonomer is selected from the group consisting of divinylbenzene, vinylbenzenesulfonic acid, acrylic acid, (meth)acrylonitrile, vinylsulfonamide, N-alkyl vinylsulfonamide N,N-dialkyl vinylsulfonamide, and 2-hydroxyethyl 2-methylprop-2-enoate (HEMA). Even more preferably, the comonomer is selected from the group consisting of divinylbenzene and vinylbenzenesulfonic acid. Most preferably, both divinylbenzene and vinylbenzenesulfonic acid are provided along with the monomer of general formula (I).

In the context of this invention, divinylbenzene can be either 1,2-diethenylbenzene, 1,3-diethenylbenzene, or 1,4-diethenylbenzene, or mixtures thereof. 1,4-diethenylbenzene or mixtures comprising 1,4-diethenylbenzene are preferred because these provide a more spacious crosslink, which improves the solvent-permeability of the resulting polymer.

In the context of this invention, vinylbenzenesulfonic acid can be either 2-vinylbenzenesulfonic acid, 3-vinylbenzenesulfonic acid, or 4-vinylbenzenesulfonic acid, or mixtures thereof. 4-vinylbenzenesulfonic acid is preferred because of its desirable polymerization kinetics. Vinylbenzenesulfonic acid can be provided as a salt, such as sodium vinylbenzenesulfonic acid, or calcium (vinylbenzenesulfonic acid)₂. The provision of salts of vinylbenzenesulfonic acid can improve the solubility of this comonomer and is particularly suitable when polymerization takes place in aqueous or otherwise highly polar media.

For crosslinked sorbents, it is preferred that at least one crosslinking comonomer is provided along with the monomer of general formula (I). A crosslinking comonomer generally has more than one reactive moiety that can participate in the polymerization reaction. Preferably, such a crosslinking comonomer is selected from the group consisting of: divinylbenzene, a telechelic N,N′-alkylenebisacrylamide such as N,N-methylenebisacrylamide (NMAA), divinyl sulfone, and butadiene, preferably divinylbenzene is provided along with the monomer of general formula (I). Alternately, the polymer can be crosslinked after the polymerization of step ii), for example by reacting polymer chains with one another, possibly via side chains of comonomers. As such, in preferred embodiments the invention provides a production method according to the invention, wherein the polymer is crosslinked after polymerization or during polymerization. Preferably, the polymer is crosslinked during polymerization, more preferably using a crosslinking comonomer.

In the context of this invention, the amount of crosslinking is defined as the amount of crosslinking comonomers that was present in the polymerization mixture during step ii). A larger amount of crosslinking results in more dense sorbents; a smaller amount of crosslinking results in more porous or more macroporous sorbents. Preferably, at most 10% crosslinking comonomer is used. More preferably, at most 5% crosslinking comonomer is used. Even more preferred, for crosslinked sorbents, from 0.1% to 5% crosslinking comonomer is used, more preferably 0.2% to 4% crosslinking comonomer is used, even more preferably 0.4% to 4% crosslinking comonomer is used, most preferably 0.8% to 3% crosslinking comonomer is used, such as about 1% to about 2%, or about 2%.

In preferred embodiments, less than 80% crosslinking comonomer is used. In more preferred embodiments, less than 67% crosslinking comonomer is used. In highly preferred embodiments, less than 50% crosslinking comonomer is used. In most preferred embodiments, less than 10% crosslinking comonomer is used. Accordingly, in preferred embodiments 1% to 10% crosslinking comonomer is used; in more preferred embodiments 2% to 10% crosslinking comonomer is used; in even more preferred embodiments 2% to 8% crosslinking comonomer is used; in still more preferred embodiments 2% to 7% crosslinking comonomer is used; in most preferred embodiments 3% to 6% crosslinking comonomer is used.

A sorbent comprising a hydrophilic comonomer is referred to herein as a hydrophilic sorbent. A hydrophilic comonomer enables aqueous solvents to more easily permeate the sorbent, so that nucleophilic waste solutes can similarly more easily permeate the sorbent. This allows the interior of the sorbent to also participate in the binding of nucleophilic waste solutes. There is a balance, because hydrophilic comonomers generally cannot bind nucleophilic waste solutes in the way that PGA-type moieties can. Thus, the increase in hydrophilic comonomer content makes PGA-type moieties more effective, but reduces their number.

For hydrophilic sorbents, it is preferred that at least one hydrophilic comonomer is provided along with the monomer of general formula (I). Preferably, such a hydrophilic comonomer is selected from the group consisting of: vinylbenzenesulfonic acid, acrylic acid, methacrylic acid, methyl acrylate, methyl methacrylate, ethyl acrylate, ethyl methacrylate, 2-hydroxyethyl 2-methylprop-2-enoate (HEMA), 2-hydroxypropyl 2-methylprop-2-enotate, 2-hydroxyethyl prop-2-enoate, 2-hydroxypropyl prop-2-enotate, N-(2-hydroxyethyl)methacrylamide, N-(2-hydroxypropyl)methacrylamide (HPMA), N-(2-hydroxyethyl)acrylamide, N-(2-hydroxypropyl)acrylamide, and N-isopropylacrylamide (NIPAm), more preferably from the group consisting of: vinylbenzenesulfonic acid, acrylic acid, methacrylic acid, 2-hydroxyethyl 2-methylprop-2-enoate (HEMA), 2-hydroxypropyl 2-methylprop-2-enotate, 2-hydroxyethyl prop-2-enoate, 2-hydroxypropyl prop-2-enotate, N-(2-hydroxyethyl)methacrylamide, N-(2-hydroxypropyl)methacrylamide (HPMA), N-(2-hydroxyethyl)acrylamide, and N-(2-hydroxypropyl)acrylamide, preferably vinylbenzenesulfonic acid is provided along with the monomer of general formula (I). A skilled person will understand that some comonomers such as methyl methacrylate can easily be postmodified to become hydrophilic through hydrolysis of their ester. Preferably, at most 60% hydrophilic comonomer is used. More preferably, at most 50% hydrophilic comonomer is used. Even more preferred, for hydrophilic sorbents, from 0% to 50% hydrophilic comonomer is used, more preferably 0% to 40% hydrophilic comonomer is used, even more preferably 5% to 40% hydrophilic comonomer is used, more preferably still 10% to 35% hydrophilic comonomer is used, even more preferably still 15% to 35% hydrophilic comonomer is used, most preferably 20% to 30% hydrophilic comonomer is used, such as about 25%.

Step iii)—Conversion of Precursor Monomers

The polymer obtained in step ii) contains monomers of general formula (I), and these monomers can be precursor monomers that are not already PGA-type monomers. In step iii) the precursor monomers comprised in the polymer obtained in step ii) are converted into PGA-type monomers by performing a conversion reaction. A conversion reaction can comprise 1, 2, or 3 steps. In preferred embodiments, the invention provides the production method according to the invention, wherein step iii) comprises converting polymerized monomers of general formula (I) that are not PGA-type monomers into PGA-type monomers using 1, 2, or 3 reaction steps, preferably using 1 or 2 reaction steps, more preferably using 1 reaction step. When a monomer of general formula (I) already comprises a PGA-type moiety, step iii) is effectively absent if no other monomers of general formula (I) are present.

General methods for converting molecules such as those of general formula (I) are known in the art, and a skilled person can select which reactions are suitable for converting any particular monomer of general formula (I) into a PGA-type monomer. Conversion reactions depend on which monomer of general formula (I) is to be converted. Accordingly, in preferred embodiments the invention provides a production method according to the invention, wherein in step iii) a conversion reaction is used comprising a step selected from the group consisting of:
a) halogenation, preferably using hydrohalic acid or Br₂, Cl₂, or I₂;
b) oxidation, preferably using dimethyl sulfoxide (DMSO) or ethyl acetoacetate or an oxide such as SeO₂; and
c) hydrolysis, preferably by heating to about 80° C. in an aqueous environment.

Preferred oxidation methods are the Swern oxidation, which uses DMSO and oxalyl chloride and a base, for example a trialkylamine such as trimethylamine; Dess-Martin oxidation, for example using Dess-Martin periodinane; Corey-Kim oxidation, for example using N-halosuccinimide such as N-chlorosuccinimide, and dimethyl sulfide, and a base, for example a trialkylamine such as trimethylamine; Oppenauer oxidation, for example using aluminium isopropoxide and optionally multivalent iodine species; Kornblum oxidation, for example using DMSO and oxalyl chloride and a base, for example a trialkylamine such as trimethylamine, or which omits the base when it follows a halogenation; oxidation using halogen species such as iodine species, preferably using multivalent iodine species; or direct oxidation using oxides such as SeO₂, OsO₄, or MnO₂ (see Jong et al., 2019, ACS Omega, DOI: 10.1021/acsomega.9b01177); microwave-assisted direct oxidation using oxides such as SeO₂, OsO₄, or MnO₂, as described by Marminon et al., (2015, DOI: 10.1016/j.tetlet.2015.02.086). A highly preferred oxidation method is Kornblum oxidation following halogenation, using dimethyl sulfoxide (DMSO) or ethyl acetoacetate, and a hydrohalic acid such as HBr, HI, or HCl, preferably using DMSO and a hydrohalic acid such as HBr.

Halogenation can be performed using methods known in the art. Preferred methods use Br₂, Cl₂, or I₂ or N-halosuccinimides such as N-bromosuccinimide, N-chlorosuccinimide, or N-iodosuccinimide, or hydrohalic acid optionally in the presence of DMSO. In preferred embodiments, halogenation is done using Br₂, Cl₂, or I₂. In other preferred embodiments, halogenation is done using N-halosuccinimides such as N-bromosuccinimide, N-chlorosuccinimide, or N-iodosuccinimide. Halogenation can also be advantageously performed using hydrohalic acid in DMSO, which allows one-pot subsequent oxidation via the Kornblum oxidation.

Hydrolysis can be performed using any known method for hydrolysis of ketals, thioketals, aminals, and hemi variants thereof. Preferred methods for hydrolysis are by heating to about 80° C. in an aqueous environment, preferably in the presence of catalytic amounts of acid. Hydrolysis is preferably not in a basic environment.

More preferably, in step iii) a conversion reaction is used comprising a step selected from the group consisting of:
a) halogenation, preferably using hydrohalic acid in DMSO at about 80° C.;
b) oxidation, preferably using dimethyl sulfoxide (DMSO) at about 80° C.; and
c) hydrolysis, preferably by heating to about 80° C. in an aqueous environment, more preferably in the presence of catalytic acid.

Most preferably, steps a) and b) of the above are comprised. This is particularly useful for (I-Ac) type monomers. The skilled person will understand that various steps as described above are mechanistic steps and can be performed simultaneously, such as by performing an oxidation using DMSO at 80° C. in the presence of a halogen source such as molecular dihalogen, hydrohalic acid, or N-halogen-succinimide, resulting in both halogenation and oxidation.

Particularly, monomers of general formula (I-PGA) or (I-PGAH) generally do not need conversion as they already comprise a PGA type moiety; monomers of general formula (I-PGAH) can be dehydrated to form monomers of general formula (I-PGA), and monomers of general formula (I-PGA) can be hydrated to form monomers of general formula (I-PGAH). Such dehydration is preferably via incubation in a dry solvent, more preferably at raised temperature such as above 60 or 80 or 100° C. Hydration occurs spontaneously in the presence of water, preferably at ambient conditions.

Particularly, monomers of general formula (I-Ac) are generally successfully converted into PGA-type monomers in two steps in a single pot, using dimethyl sulfoxide (DMSO) and hydrohalic acid such as HCl, HBr, or HI. In such a reaction, the acetyl moiety is first halogenated, after which it is oxidized by the DMSO. When for example polymerized VPE is used in this reaction, this one-pot reaction directly leads to PGA-type sorbents. After such conversion the sorbent is preferably washed such as with water, more preferably until the pH of the washes is in the range of pH 5-9. Accordingly, in preferred embodiments, the invention provides the production method according to the invention, wherein step i) comprises the provision of a monomer of general formula (I-Ac), and wherein step iii) comprises converting polymerized monomers of general formula (I-Ac) into PGA-type monomers using dimethyl sulfoxide (DMSO) and hydrohalic acid, preferably using DMSO and a hydrohalic acid such as HBr.

Particularly, monomers of general formula (Ih) wherein at least one of h¹ and h² is a halogen are generally successfully converted into PGA-type monomers in a single oxidation step, preferably using dimethyl sulfoxide (DMSO). This is analogous to the conversion for general formula (I-Ac) described above, omitting the halogenation. Accordingly, in preferred embodiments, the invention provides the production method according to the invention, wherein step i) comprises the provision of a monomer of general formula (Ih) wherein at least one of h¹ and h² is a halogen, and wherein step iii) comprises converting polymerized monomers of general formula (Ih) into PGA-type monomers using dimethyl sulfoxide (DMSO). Alternately, monomers of general formula (Ih) wherein h¹ and h² together form a single moiety (such as a ketal) or form a (hemi)ketal, (hemi)thioketal, or (hemi)aminal, can be converted into PGA-type monomers using hydrolysis at elevated temperatures such as at 80° C., preferably using catalytic acid such as about 1 vol.-% acetic acid.

In preferred embodiments, the invention provides the production method according to the invention, wherein in step iii) more than 30%, 35%, 40%, 45%, 50%, 55%, 60%, 65%, 70%, 75%, 80%, 85%, 90%, 95%, or 100% of the monomers of general formula (I) are converted into PGA-type monomers. In more preferred embodiments, the invention provides the production method according to the invention, wherein in step iii) more than 55%, 60%, 65%, 70%, 75%, 80%, 85%, 90%, 95%, or 100% of the monomers of general formula (I) are converted into PGA-type monomers. In even more preferred embodiments, the invention provides the production method according to the invention, wherein in step iii) from 55% to 100% of the monomers of general formula (I) are converted into PGA-type monomers. In most preferred embodiments, the invention provides the production method according to the invention, wherein in step iii) from 55% to 90% of the monomers of general formula (I) are converted into PGA-type monomers.

In particular embodiments of the invention, in step iii) 0% of the monomers of general formula (I) are converted into PGA-type monomers. This is particularly the case when all polymerized monomers of general formula (I) already are PGA-type monomers.

Composition and Other Products

In a third aspect of the invention, the invention provides a composition comprising the PGA-type sorbent according to the invention and a pharmaceutically acceptable excipient. Such a composition is referred to hereinafter as a composition according to the invention. Such a composition is preferably a pharmaceutically composition.

Compositions and pharmaceutical compositions according to the invention may be manufactured by processes well known in the art; e.g., by means of conventional mixing, dissolving, granulating, dragee-making, levigating, emulsifying, encapsulating, entrapping or lyophilizing processes, which may result in liposomal formulations, coacervates, oil-in-water emulsions, nanoparticulate/microparticulate powders, or any other shape or form. Compositions for use in accordance with the invention thus may be formulated in conventional manner using one or more physiologically acceptable carriers comprising excipients and auxiliaries that facilitate processing of the active compounds into preparations which can be used pharmaceutically. Proper formulation is dependent on the route of administration chosen.

Oral and parenteral administration may be used wherein the compound or composition according to the invention can be formulated readily by combining a compound or composition according to the invention with pharmaceutically acceptable carriers well known in the art, or by using a compound or composition according to the invention as a food additive. Such strategies enable the compounds or compositions according to the invention to be formulated as tablets, pills, dragees, capsules, liquids, gels, syrups, slurries, suspensions and the like, for oral ingestion by a subject to be treated. Preparations or pharmacological preparations for oral use can made with the use of a solid excipient, optionally grinding the resulting mixture, and processing the mixture of granules, after adding suitable auxiliaries, if desired, to obtain tablets or dragée cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as cross-linked polyvinyl pyrrolidone, agar, or alginic acid or a salt thereof such as sodium alginate. Additionally, coformulations may be made with uptake enhancers known in the art.

Alternatively, one or more components of the composition may be in powder form for constitution with a suitable vehicle, e.g., sterile pyrogen-free water, before use. Components of the composition may be supplied separately.

The compositions or pharmaceutical compositions according to the invention also may comprise suitable solid or gel phase carriers or excipients. Examples of such carriers or excipients include but are not limited to calcium carbonate, various sugars, starches, cellulose derivatives, gelatin, and polymers such as polyethylene glycols.

A pharmaceutical composition according to the invention can also comprise a further pharmaceutically active substance, preferably a further pharmaceutically active substance for the treatment of a disease or condition associated with accumulation of urea or with improper clearance of urea, such as acute kidney failure or end stage kidney disease (ESKD).

A composition according to the invention or a PGA-type sorbent according to the invention can advantageously be used in renal replacement therapy, such as peritoneal dialysis or hemodialysis. During such use, the sorbent or composition is generally present in a cartridge or membrane, which can be replaceably inserted in a (hemo)dialysis device or for example a peritoneal dialysis device. Accordingly, the invention provides a cartridge for use in a dialysis device, comprising a PGA-type sorbent according to the invention, or comprising a composition according to the invention. Such a dialysis device can be a hemodialysis device or a device for regeneration of peritoneal dialysate in peritoneal dialysis. Accordingly, the invention provides a membrane for use in a dialysis device, comprising a PGA-type sorbent according to the invention, or comprising a composition according to the invention. Such a dialysis device can be a hemodialysis device or a device for regeneration of peritoneal dialysate in peritoneal dialysis. Accordingly, the invention provides a dialysis device comprising a PGA-type sorbent according to the invention, a composition according to the invention, or a cartridge according to the invention. Such a dialysis device can be a hemodialysis device or a device for regeneration of peritoneal dialysate in peritoneal dialysis.

Besides the PGA-type sorbent according to the invention, or the composition according to the invention, as comprised in the cartridge, the membrane, or the dialysis device, such cartridges, membranes, and dialysis devices are known in the art. In particular embodiments, the cartridge is a disposable cartridge. In particular embodiments, the cartridge is a regenerable cartridge. Cartridges can also be referred to as cassettes. The cartridge is preferably adaptable to be used with various different types of components and to be arranged in a variety of ways. A cartridge may comprise further sorbents. By removing nucleophilic waste solutes, the cartridge at least partially regenerates the dialysate and/or filtrate used during dialysis. The cartridge preferably includes a body having a fluid inlet and a fluid outlet. The interior of the cartridge is preferably constructed and arranged so that fluid entering the interior from the inlet flows through the sorbent and subsequently through the outlet.

A membrane for use in a dialysis device is preferably semipermeable. It can be sheet-like and separate two volumes by acting as a wall or as part of a wall. It can be in the form of a fibre bundle that connects two volumes. A very suitable fibre bundle is described in WO2006019293, wherein a bundle of hollow or solid fibres having multiple porous layers concentrically arranged is described. In such a bundle one of the layers can comprise functionalized or active particles that are well accessible to the fluids that flow through the membrane. In preferred embodiments of the invention, the PGA-type sorbent according to the invention is comprised in a membrane such as a bundle of fibres wherein the fibres have multiple concentric layers, preferably comprised in one of the layers of such a fibre, preferably configured in such a way that it comes into contact with fluids passing through the membrane, or with fluids that pass through the membrane, preferably further configured to bind nucleophilic waste solutes in said fluids. Such membranes can act as combined membrane and sorbent, allowing further miniaturization.

A dialysis device is a closed, sterile system. It comprises one or two fluid circuits. It usually comprises two circuits: a so-called patient loop, which is a fluid circuit that is arranged for a subject's fluid such as blood or peritoneal dialysate to flow through it, and a so-called regeneration loop, wherein a dialysis fluid such as dialysate and/or filtrate is circulated through a cartridge as described above. The two circuits are separated from each other by a (semi-permeable) membrane, through which waste solutes can diffuse or pass from the subject's fluid into the dialysis fluid. Air, moisture, pathogens, and fluids from the environment around the dialysis device cannot enter into the fluid circuits. The dialysis system only permits fluids (such as ultrafiltrate) and air to exit or enter these fluid circuits under controlled circumstances.

Medical Use

In a fourth aspect the invention provides the medical use of PGA-type sorbents according to the invention, and of compositions according to the invention. As such, this aspect provides a PGA-type sorbent according to the invention, or composition according to the invention, for use as a medicament, preferably for use in the treatment of a disease or condition associated with accumulation of urea or with improper clearance of urea. Such a sorbent or composition is referred to herein as a product for use according to the invention.

In particular embodiments of this aspect, the invention provides a PGA-type sorbent according to the invention, or a composition according to the invention, for use as a medicament for use in the treatment of a disease or condition associated with accumulation of ammonia or with improper clearance of ammonia. In further particular embodiments of this aspect, the invention provides a PGA-type sorbent according to the invention, or a composition according to the invention, for use as a medicament, wherein the PGA-type sorbent is for binding urea. In further particular embodiments of this aspect, the invention provides a PGA-type sorbent according to the invention, or a composition according to the invention, for use as a medicament, wherein the PGA-type sorbent is for binding ammonia.

Treatment of a disease or condition can be the amelioration, suppression, prevention, delay, cure, or prevention of a disease or condition or of symptoms thereof, preferably it shall be the suppression of symptoms of a disease or condition. Urea can accumulate or can be insufficiently cleared in case of kidney failure. Examples of diseases or conditions associated with accumulation of urea or with improper clearance of urea are end stage kidney disease (ESKD); severe acute kidney failure; increased hepatic production of urea for example due to gastro-intestinal haemorrhage; increased protein catabolism, for example due to trauma such as major surgery or extreme starvation with muscle breakdown; increased renal reabsorption of urea, for example due to any cause of reduced renal perfusion, for example congestive cardiac failure, shock, severe diarrhea; iatrogenic conditions due to urea infusion for its diuretic action, due to drug therapy leading to an increased urea production such as treatment with tetracyclines or corticosteroid; chronic kidney failure; and urinary outflow obstruction.

Products for use according to the invention can be administered to a subject in need thereof, allowing the product for use according to the invention to bind nucleophilic waste solutes in the subject. Such administration is preferably administration of an effective amount. The use of other sorbents in such a method is known in the art (Gardner et al., Appl Biochem Biotechnol. 1984; 10:27-40.)

Administration can be via methods known in the art, preferably via oral ingestion in any formulation known in the art such as a capsule, pill, lozenge, gel capsule, push-fit capsule, controlled release formulation, or via rectal administration as a clyster or suppository. It can be once per week, 6, 5, 4, 3, 2, 1 time per week, daily, twice daily, or three times per day, or four times per day.

Products for use according to the invention are suitable for use in a method of treatment. Such a method of treatment can be a method comprising the step of administering to a subject, preferably a subject in need thereof, an amount, preferably an effective amount, of product for use according to the invention.

With respect to dialysis therapy, the present invention can be used in a variety of different dialysis therapies to treat kidney failure. Dialysis therapy as the term or like terms are used throughout the text is meant to include and encompass any and all forms of therapies to remove waste, toxins and excess water from the subject suffering from a disease or condition. The hemo therapies, such as hemodialysis, hemofiltration and hemodiafiltration, include both intermittent therapies and continuous therapies used for continuous renal replacement therapy (CRRT). The continuous therapies include, for example, slow continuous ultrafiltration (SCUF), continuous venovenous hemofiltration (CVVH), continuous venovenous hemodialysis (CVVHD), continuous venovenous hemodiafiltration (CVVHDF), continuous arteriovenous hemofiltration (CAVH), continuous arteriovenous hemodialysis (CAVHD), continuous arteriovenous hemodiafiltration (CAVHDF), continuous ultrafiltration periodic intermittent hemodialysis or the like. The present invention can also be used during peritoneal dialysis including, for example, continuous ambulatory peritoneal dialysis, automated peritoneal dialysis, tidal peritoneal dialysis, intermittent peritoneal dialysis, continuous flow peritoneal dialysis, flow-through peritoneal dialysis, and the like. Further, although the present invention, in an embodiment, can be utilized in methods providing a dialysis therapy for subjects having acute or chronic kidney failure or disease, it should be appreciated that the present invention can also be used for acute dialysis needs, for example, in an emergency room setting. However, it should be appreciated that the compositions of the present invention can be effectively utilized with a variety of different applications, physiologic and non-physiologic, in addition to dialysis.

Method of Use

The PGA-type sorbents according to the invention are surprisingly effective at binding nucleophilic solutes, preferably nucleophilic waste solutes. In a fifth aspect, the invention provides a method for removing nucleophilic waste solutes from a fluid, comprising the steps of:
i) providing a fluid comprising nucleophilic waste solutes, and
iia) contacting said fluid with a PGA-type sorbent according to the invention, or with a composition according to the invention, or with a cartridge according to the invention, or alternately
iib) contacting said fluid with a dialysis fluid through a membrane, wherein the dialysis fluid is in contact with a PGA-type sorbent according to the invention, or with a composition according to the invention, or with a cartridge according to the invention, and
iii) optionally, recovering the fluid.

Such a method is referred to hereinafter as a binding method according to the invention. The method can be a continuous process, wherein provision of a fluid comprising nucleophilic waste solutes is through provision of a continuous flow of fluid. In such a case, preferably step iii) is not optional, and is also continuously performed. A binding method according to the invention always comprises step i), a step ii) (one of either step iia) or step iib)), and optionally step iii).

Nucleophilic waste solutes are dissolved substances that are nucleophilic, the removal of which is desired. For example, in human blood, urea is a waste solute. In unpurified water intended as drinking water, most organic nucleophiles are waste solutes. Examples of nucleophilic waste solutes are ammonia, urea, creatinine, and small molecule organic amines, thiols, or alcohols. The chemical binding properties make the PGA-type sorbent according to the invention well suited for a variety of different applications subject to physiological and/or non-physiological conditions. In an embodiment, the PGA-type sorbent according to the invention can be used to remove metabolic waste, such as urea, creatinine, uric acid and/or others like uremic toxins, biological matter, proteinaceous matter, and/or the like from blood, peritoneal dialysate, and/or solutions used to dialyze and/or filter blood, such as dialysate and/or filtrate. Due to its relevance as described elsewhere herein, highly preferred nucleophilic waste solutes are urea and ammonia. In preferred embodiments, the nucleophilic waste solute is ammonia. In other preferred embodiments, the nucleophilic waste solute is urea.

In step i) a fluid comprising a nucleophilic waste solute is provided. This can be waste water which is to be purified, it can be waste solvent which is to be purified, but it can also be a (body) fluid from a subject, such as blood or peritoneal dialysate. When the fluid of step i) is a fluid from a subject, it is preferably blood or peritoneal dialysate, most preferably blood, and preferably a fluid that has been previously obtained from a subject.

In step ii), two options exist. In one option, step iia), the fluid itself is directly contacted with the PGA-type sorbent according to the invention, the composition according to the invention, or the membrane or cartridge according to the invention. Step iia) is very suitable for purification of solvents, or for fluids that are not intended for consumption or for medical purposes after removal of the waste solutes. Step iib) separates the binding sorbent from fluid provided in step i) by using dialysis fluid and/or filtrate. Step iib) is particularly suited for the removal of nucleophilic waste solutes from pharmaceutical solutions or from fluids obtained from a subject, such as bodily fluids of a subject. Contacting preferably lasts 24 hours, 12 hours, 6 hours, 4 hours, 3 hours, 2 hours, 60 minutes, 50 minutes, 40 minutes, 30 minutes, 20 minutes, 15 minutes, 10 minutes, 9, 8, 7, 6, 5, 4, 3, 2, or 1 minutes, or less. Contacting can also be in continuous flow past the sorbent, in which case the total amount of removed waste solute is more relevant.

Membranes for use in step iib) are preferably semipermeable membranes. These are known in the art, and can for example be the semipermeable membrane present in a conventional (hemo)dialysis device. Membranes according to the invention as described above can also be used. Dialysis fluids are known in the art and can range from ultrapure water to physiological buffers. Non-limiting examples of dialysis fluids are media comprising known amounts of for example Na, K, Ca, Mg, Cl, acetate, HCO₃, and glucose, such as those available from MDN Neubrandenburg GmbH (Neubrandenburg, Germany), or from Baxter (Deerfield, Ill., USA), or from Dirinco B. V. (Oss, the Netherlands).

In step iii), which is optional, the fluid is recovered. The PGA-type sorbent according to the invention is often porous, macroporous, or swellable in aqueous media, such that fluids can flow through it and permeate it. Recovery of a fluid that has been contacted with a sorbent is readily achieved by filtration, centrifugation, or removal of the cartridge containing the sorbent. Recovery of a fluid allows its further processing, or its return to a subject. In preferred embodiments within this aspect, the fluid is recovered.

Preferably, when a fluid is recovered in step iii), relevant physiological parameters are subsequently analyzed and adjusted when appropriate. Examples are ion concentrations, osmolality, pH, particularly Na concentration, Ca concentration, and Mg concentration. Accordingly, a preferred step iii) is the step of recovering the fluid, after which at least one of fluid pH, fluid sodium concentration, fluid magnesium concentration, and fluid calcium concentration is determined and optionally adjusted to a reference value. Preferred reference values are corresponding physiological values for the fluid type. The adjustment can be done in any suitable way known in the art. The adjustment is preferably performed when a deviation from the reference value is detected.

In preferred embodiments of the binding method, at least 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.67, 1.68, 1.69, 1.70, 1.71, 1.72, 1.73, 1.74, 1.75, 1.76, 1.77, 1.78, 1.79, 1.80, 1.85, 1.90, 1.95, 2.00, 2.05, 2.10, 2.15, 2.20, 2.25, 2.30, 2.35, 2.40, 2.45, or 2.50 mmol of nucleophilic waste solute per gram of sorbent is removed; preferably at least 1.55, 1.56, 1.57, 1.58, 1.59, 1.60, more preferably at least 1.60, even more preferably at least 1.80, most preferably at least 2.10, 2.15, 2.20, 2.25, 2.30, 2.35, 2.40, 2.45, or 2.50 mmol of nucleophilic waste solute per gram of sorbent is removed. This removal preferably entailed removal of at least 10%, 20%, 30%, 40%, 50%, 60%, 70%, 80%, 85%, 90%, 91%, 92%, 93%, 94%, 95%, or more of a particular nucleophilic waste solute's initial concentration from a fluid as provided in step i), more preferably removal of at least 50% or more.

### General Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value more or less 5% of the value.

Molecules provided in this invention can be optionally substituted. Suitable optional substitutions are replacement of —H by a halogen. Preferred halogens are F, Cl, Br, and I. Further suitable optional substitutions are substitution of one or more —H by —NH₂, —OH, ═O, alkyl, alkoxy, haloalkyl, haloalkoxy, alkene, haloalkene, alkyn, haloalkyn, and cycloalkyl. Alkyl groups have the general formula CₙH₂ₙ₊₁ and may alternately be linear or branched. Unsubstituted alkyl groups may also contain a cyclic moiety, and thus have the concomitant general formula CₙH₂n−1. Optionally, the alkyl groups are substituted by one or more substituents further specified in this document. Examples of alkyl groups include methyl, ethyl, propyl, 2-propyl, t-butyl, 1-hexyl, 1-dodecyl, etc.

Unless stated otherwise, —H may optionally be substituted with one or more substituents independently selected from the group consisting of C₁-C₁₂ alkyl groups, C₂-C₁₂ alkenyl groups, C₂-C₁₂ alkynyl groups, C₃-C₁₂ cycloalkyl groups, C₅-C₁₂ cycloalkenyl groups, C₈-C₁₂ cycloalkynyl groups, C₁-C₁₂ alkoxy groups, C₂-C₁₂ alkenyloxy groups, C₂-C₁₂ alkynyloxy groups, C₃-C₁₂ cycloalkyloxy groups, halogens, amino groups, oxo and silyl groups, wherein the silyl groups can be represented by the formula (R²)₃Si—, wherein R² is independently selected from the group consisting of C₁-C₁₂ alkyl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ alkynyloxy groups and C₃-C₁₂ cycloalkyloxy groups, wherein the alkyl groups, alkenyl groups, alkynyl groups, cycloalkyl groups, alkoxy groups, alkenyloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S.

When a structural formula or chemical name is understood by the skilled person to have chiral centers, yet no chirality is indicated, for each chiral center individual reference is made to all three of either the racemic mixture, the pure R enantiomer, and the pure S enantiomer. When two moieties are said to together form a bond, this implies the absence of these moieties as atoms, and compliance of valence being fulfilled by a replacing electron bond. All this is known in the art.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use are suitable for use in methods of treatment.

Throughout this application, when percentages are used for expressing amounts of monomers and comonomers in a mixture, mole percentages are intended, unless stated otherwise or explicitly plain from context. Throughout this application, (hemo)dialysis refers to both hemodialysis and dialysis. In general, a dialysis device can refer to any type of dialysis device as described herein.

The present invention has been described above with reference to a number of exemplary embodiments. Modifications and alternative implementations of some parts or elements are possible, and are included in the scope of protection as defined in the appended claims. All citations of literature and patent documents are hereby incorporated by reference.

### DESCRIPTION OF DRAWINGS

FIG. 1—Reaction of the PGA/PGAH-derivatives with urea. In the case of a PGA-type sorbent, R can be a polymeric backbone.

FIG. 2A—Synthesis of PGA-type sorbents from styrene. R¹=optional crosslinker, R²=unmodified styrene, R³=side product of conversion reaction.

FIG. 2B—Synthesis of PGA-type sorbents from a precursor monomer (VPE). R¹=optional crosslinker, R²=optional side product of conversion reaction.

FIG. 3—Oxidation of PS-Ac or of pVPE using HBr and DMSO. PGA=Phenylglyoxaldehyde, PGAH=phenylglyoxaldehyde hydrate, PGOA=phenylglyoxilic acid.

FIG. 4—The urea binding capacity of pVPE beads (table 2 entry 2) as a function of oxidation time of pVPE. Conditions for oxidation: pVPE (500 mg) in DMSO (5.0 mL) and 48% aqueous HBr (1.45 mL) stirred with a Teflon blade stirrer at 80° C. for 4-12 hours. Per timepoint ±150 mg beads were removed from the suspension and tested for urea binding.

FIG. 5—IR spectra of PGAH, PS-AC-Ox@urea, pVPE-Ox-(4)@urea and the 2:1 addition product of PGAH and urea. The adduct, indicated as 3′a, was synthesized as described in Jong, J. A. W. et al., ACS Omega 2019, 4 (7), 11928-11937.

FIG. 6A—Urea binding of PS—Ac-Ox and pVPE-Ox-(4) in time. Expressed in mmol urea/g sorbent.

FIG. 6B—Relative urea binding (percentage of the maximum binding capacity). Conditions: sorbent (10 mg/mL) in 30 mM urea solution in PBS at 37° C. (N=4). Triangles represent pVPE-Ox, squares represent PS—Ac-Ox.

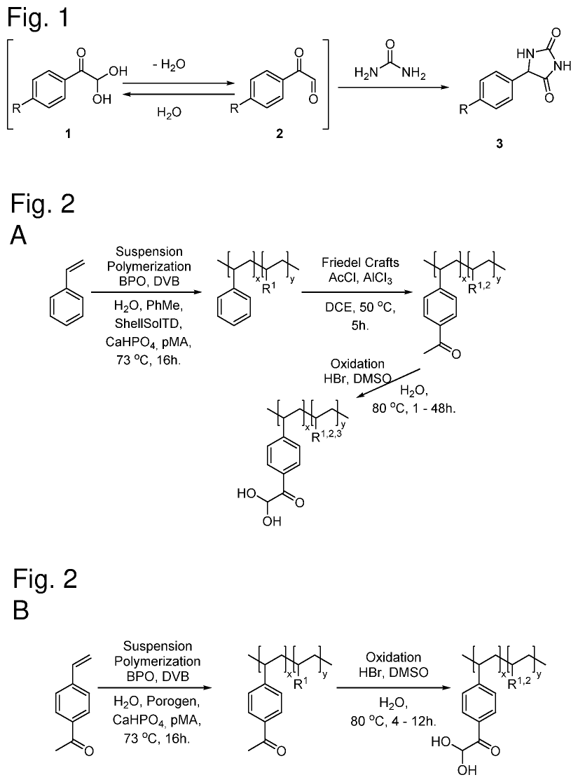

### EXAMPLES

### Example 1—Materials and Methods

### 1.1 NMR, UV and IR Spectroscopy

NMR spectra were recorded on a Bruker 600 MHz with a BBI probe at room temperature (RT). Residual solvent signals were used as internal standard (1H: δ 7.26 ppm, 13C (1H): δ 77.16 ppm for CDCl3). Chemical shifts (δ) are given in ppm and coupling constants (J) are given in hertz (Hz). Resonances are reported as s (singlet), d (doublet), t (triplet), q (quartet), bs (broad singlet) and m (multiplet) or combinations thereof. UV absorption spectra were recorded in triplicate with a BMG LABTECH SpectroStar Nano plate reader using a UV-Star Microplate 96 well obtained from Greiner Bio-One (Alphen aan de Rijn, the Netherlands). Infrared (IR) spectra were recorded neat using a Perkin Elmer ATRU Spectrum 2.

### 1.2 Determination of Pseudo-First Order Rate Constants

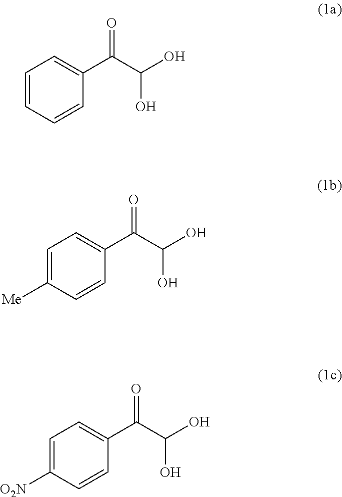 [SMILES: O=C(c1ccccc1)C(O)O ; Cc1ccc(C(=O)C(O)O)cc1 ; O=C(c1ccc([N+](=O)[O-])cc1)C(O)O]

PGAH (1a) and two PGAH derivatives (1b and 1c) (0.3 mmol, 1.0 eq.) were dissolved in 1:1 v/v mixture of PBS:dimethylsulfoxide (DMSO) (10 mL). Urea (901 mg, 15 mmol, 50 eq.) was dissolved in the PGAH solution which was subsequently magnetically stirred at 50° C. Samples (50 μL) from the reaction mixture were taken at different time points, diluted 10 (1a) or 15 (1b and 1c) times with 1:1 v/v DMSO:PBS (500 or 700 μL) and subsequently diluted another 10× using the same solvent mixture (thus resulting in a final 100 or 150 times dilution, respectively). The concentrations of the PGAH (derivatives) 1a-c in the 100 or 150 times diluted samples were determined by UV spectroscopy (260, 263 and 270 nm, for 1a, 1b and 1c, respectively). A calibration curve was prepared using a dilution series in 1:1 v/v mixture of PBS:DMSO (final concentrations varied from 0.030-0.360 mM) from a stock solution of the PGAH (derivatives) (30 mM) in 1:1 (v/v) DMSO:PBS. The k_{PFO}-values for the PGAH analogues were determined from the slopes of the plots of log [PGAH] versus time.

### 1.3 Preparation of 10% Polymethacrylic Acid Sodium Salt Solution in Water

In a glass reactor equipped with mechanical stirrer, polymethacrylic acid (10 grams) was dissolved in water (84 mL) by heating to 80° C. and stirring for 30 minutes. Next an aqueous 50% NaOH (2.67 mL; 68 mmol NaOH) solution was added and stirring was continued for 60 minutes at the same temperature. The obtained viscous solution was transferred into a Falcon tube and stored at 4° C. for later use as thickening agent of the aqueous phase in suspension polymerization.

### 1.4 Suspension Polymerization of Styrene

For the suspension polymerization of styrene we essentially used a method as described by Jong (Jong, G. J. D. Ion exchangers from poly(aminostyrene) and ethylene imine. 1971). However, ShellSolTD and a poly(methacrylic acid) sodium salt solution were used instead of hexane and polyacrylic acid sodium salt.

The detailed procedure was as follows: the aqueous phase was prepared by addition of NaCl (340 mg), poly(methacrylic acid) sodium salt solution (8.32 g of a 10% solution in water) and CaHPO₄ (3.06 g) to water (540 mL) in a glass reactor equipped with a teflon blade stirrer. The aqueous phase was stirred for 30 minutes at RT and the pH was 6.9. The organic phase was prepared by mixing styrene (229 mL, 2.0 mol), ShellSolTD (276 mL) and toluene (27 mL) in a beaker. Next, 55% technical grade divinylbenzene (DVB) (13 mL, 50 mmol, 2.5 mol %) and a 50% benzoylperoxide blend with dicyclohexyl phthalate (6.0 g, 12.4 mmol, 0.6 mol %) was added to the organic phase and stirred until the initiator was dissolved and a homogeneous solution was formed at room temperature (RT). The organic phase was subsequently added to the aqueous phase in the glass reactor under continuous mechanical stirring at 180 rpm, by which an o/w emulsion was formed, and oxygen was removed by flushing with nitrogen gas for 20 minutes. Next, the emulsion was heated at 73° C. in an oil bath for 16 hours under mechanical stirring. The resulting suspension was allowed to cool to RT and was poured over a sieve (cut-off 200 μm, Veco B. V.) and washed with acetone and water. The white beads were collected and dried over P₂O₅ under vacuum, resulting in 216 g polystyrene (PS) beads. TGA analysis showed ˜14% volatiles present, indicating a yield of solid material of ˜186 gram (86%).

Macroporous polystyrene beads (PS) were thus synthesized by suspension co-polymerization of styrene and a low content of divinylbenzene (DVB, 2.5%) in a cylindrical reactor with mechanical stirrer. A mixture of toluene and ShellSolTD@ (9:91 v/v) was used as a non-solvating porogen and spherical beads were obtained in a 97% yield. The average diameter of the beads as determined by light microscopy was 0.49±0.18 mm. Scanning Electron Microscopy (SEM) analysis showed that pores are clearly visible on the surface of the beads. The surface area (S_{BET}) and pore volume of the beads as determined by nitrogen physisorption were 36.3 m²/g and 0.32 mL/g, respectively. The plot of the pore volume versus the pore diameter showed that the pores present in the material were mainly in the range of 50-100 nm, demonstrating that the obtained beads are indeed macroporous.

### 1.5 Friedel-Crafts Acetylation of Polystyrene

In a glass reactor equipped with a teflon blade stirrer, PS beads (80.9 g, 0.77 mol aromatic groups, 1.0 eq.) were swollen in 1,2-dichloroethane (DCE, 750 mL) for 30 minutes under mechanical stirring. Anhydrous AlCl₃ (156 g, 1.17 mol, 1.5 eq.) was added portion wise (3-5 gram) to the suspension over the course of 15 minutes. After all AlCl₃ was added, acetyl chloride (66 mL, 0.94 mol, 1.2 eq.) was added slowly and the suspension was heated to 50° C. in an oil bath for 5 hours, after which the formation of HCl-gas (caused by the reaction of aromatic group and acetyl chloride) stopped. The suspension was allowed to cool to RT, after which the suspension was filtered (cut-off 200 μm). The residue was suspended in a 500 mL of 6 M HCl solution at 0° C. in an ice bath and stirred for 30 minutes to remove aluminum salts; this step was repeated twice. The suspension was filtered (cut-off 200 μm, Veco B. V.) and washed with acetone and water until the pH of the filtrate was >5. The residue was dried over P₂O₅ under vacuum, resulting in acetylated polystyrene (PS-Ac, 71.6 g).

### 1.6 Halogenation and Kurnblum Oxidation of Acetylated Polystyrene

In a glass reactor equipped with a teflon blade stirrer, PS-Ac beads (60.0 g) were swollen in DMSO (600 mL, 8.45 mol) for 30 minutes under continuous stirring, after which an aqueous solution of 48% HBr (175 mL, 1.55 mol) was slowly added. One of the outlets of the reactor was capped with a septum containing a needle allowing escape of the formed Me₂S. The suspension was stirred at 80° C. for 8 hours, after which the reaction mixture was filtered (cut-off 200 μm, Veco B. V.). The residue was washed with water until the pH of the filtrate was >5. The residue was dried over P₂O₅ under vacuum, resulting in PS—Ac-Ox (55.2 grams).

### 1.7 Scanning Electron Microscopy Analysis of Sorbent Particles

The morphology of the beads was analyzed by scanning electron microscopy (SEM, Phenom, FEI Company, the Netherlands). Dried beads were transferred onto 12-mm diameter aluminum specimen stubs (Agar Scientific Ltd., England) using double-sided adhesive tape. Prior to analysis, the beads were coated with platinum using an ion coater under vacuum. The samples were imaged using a 5 kV electron beam.

### 1.8 Determination of the Size of the Beads by Light Microscopy

The diameters of the beads were measured using optical microscopy, utilizing a size calibrated Nikon eclipse TE2000-U microscope equipped with a digital camera (Nikon DS-2Mv camera and Nikon DS-U1 digital adapter, with a 4× magnification) and the NIS-elements basic research software package. Images of the beads were taken in the dry state and for 30 arbitrary beads 3 points on the perimeter of the beads were identified to allow calculation of circular diameter by the program. The average diameters and standard deviations are reported.

### 1.9 Quantitative ¹³C Solid State NMR Analysis of the Different Beads

For solid-state ¹³C NMR measurements, beads were crushed and transferred into a 3.2 mm rotor for the magic-angle spinning (MAS) solid-state NMR analysis. The analysis of the samples was performed either on a Bruker 700 MHz wide-bore magnet with an AVANCE-III console or on a Bruker 400 MHz spectrometer. The spectra were recorded at room temperature (298 K) and using Magic Angle Spinning (MAS) frequency between 10 and 14 kHz, chosen to minimize the overlap of the signal with spinning sidebands. For the ¹³C direct excitation spectra, 300 pulses were applied with field strength of 55 kHz and 80 kHz SPINAL64. ¹H decoupling was applied during acquisition. The ¹³C T1 relaxation time for each sample was determined using inverse recovery and used to establish the repetition time for the different samples, set to 2*T1. Except for the pVPE-Ox-(4) sample, which showed a very short relaxation time of 1 s, for the other samples the T1 varied from 40 to 80 s. The NMR spectra were processed with 200 Hz line-broadening and analyzed with Bruker Topspin3.5.

### 1.10 Determination of Surface Area of the Beads Using Nitrogen Physisorption

N₂ physisorption isotherms were measured at −196° C. using a Micromeritics TriStar 3000 and TriStar II Plus apparatus. Prior to analysis, the samples were dried under vacuum for 16 hours at RT. Surface areas of the beads were determined using the Brunauer-Emmett-Teller (BET) method and the total pore volumes were derived from the amount of N₂ adsorbed at p/p₀=0.995. A Barrett-Joyner-Halenda (BJH) analysis was employed to determine pore size/volume distributions of the samples with the use of a Harkins-Jura thickness curve. Due to the shrinking of the porous polymeric beads and collapsing of the pores with increasing pressure, and subsequent expansion and with decreasing pressure, the correction of the dead volume is incorrect, as by default it assumes that the solid fraction of the sample does not change in volume with pressure. As the dead volume was determined at p/p₀≈0 and assumed constant during the measurement, the default dead volume-corrected isotherms decrease slightly with increasing pressures, which is physically meaningless. The relative deviation is largest for materials with low surface areas (<5 m²/g) and high materials volume fractions in the measurement tubes, such as for pVPE-Ox. A correction for this deformation i.e. change in dead volume with pressure was applied to these isotherms by a linear swelling function (V_{adjusted}=a·(p/p₀)+V_{original}), in which a represents the swelling factor relative to the material's volume at p/p₀≈0, until dV/d (p/p₀)>0 was achieved for all pressures. Values for a were between 1.2 and 7.2, indicating a significant deformation of these materials. The S_{BET} surface areas of the pVPE-Ox beads were calculated from the isotherms that were corrected for these volume changes as a function of pressure.

### 1.11 Determination of Urea Binding

The sorbent beads (15 mg) were dispersed with urea solution (1.5 mL, 30 mM) in PBS in Eppendorf tubes. The samples were placed in an oven at 37° C. on a rotating device. After 1, 2, 4, 8, 16 and 24 hours, two Eppendorf tubes per time point were taken and the beads were allowed to settle and the supernatant was removed. To determine the maximum binding capacity, the sorbent beads (50 mg per vial) were incubated for 24 hours at 70° C. with a urea solution (5 mL, 30 or 50 mM) in PBS in two glass vials, after which the beads were allowed to settle and the urea concentrations in the supernatants were determined with an AU 5800 routine chemistry analyzer (Beckman Coulter, Brea, Calif.) using a coupled enzyme reaction, which results in a colorimetric (570 nm) product proportional to the urea concentration.

### 1.12 Thermal Analysis of Monomer and Beads

Thermographic analysis (TGA) was done as follows. In a platinum pan the beads were heated at a rate of 10° C./minute. The weight loss during the ramp heating (and thereby the decomposition temperature) was determined on a TA Instruments TGA Q50. Differential scanning calorimetry (DSC) analysis of the different samples was done as follows. In an open aluminum pan the monomer or beads were heated from −50 till 250° C. at a rate of 10° C./minute and the heat flow was monitored. Next, the sample was quench cooled from 250 to −50° C. and subsequently heated again to 250° C. at a rate of 10° C./min. The T₉ or melting point was determined with a TA instruments Discovery DSC. For the beads, residual solvent evaporated during the first run and therefore the results of the second run are reported. For the monomer (VPE) events of the first run are reported.

### Example 2—Provision of Monomers

### 2.1 Design of Monomers of General Formula (I)

First, we investigated whether reactivity of a phenylglyoxaldehyde hydrate (PGAH)-based sorbent could be increased by appropriate substituents. The kinetics of the reaction of urea with para-methyl-PGAH (1b), a PGAH-derivative with an electron donating group (EDG), and with para-nitro-PGAH (1c), a PGAH-derivative with an electron withdrawing group (EWG) were analyzed and compared to the kinetics of unsubstituted PGAH (1a) with urea. Substituents on the meta-position were not investigated because for substituent effects on the reaction of ninhydrin-analogues with urea it is found that the position of the EDG has a marginal effect on the overall reactivity of ninhydrin-derivatives with urea (Jong, J. A. W. et al., ChemistrySelect 2018, 3 (4), 1224-1229). An excess (50 equivalents) of urea was used to limit the formation of an 1:2 urea-PGA adduct. Because the urea concentration is much higher than the PGAH concentration, its concentration stays almost constant, and thus pseudo-first order conditions are valid, making the reaction rate (−d[PGAH]/dt) dependent on the PGAH concentration only. The pseudo-first order kinetics of the reaction of PGAH (and its derivatives) with urea were analyzed by determining the concentrations of 1a-c in time using UV spectroscopy. The solvent for this reaction was a 1:1 (v/v) PBS/DMSO mixture due to the very low solubility of 1b and 1c in PBS only. The pseudo-first order rate constants (k_{PFO}) correspond with the negative slopes in the plot of the logarithm of the PGAH-(derivatives) concentration divided by log (e) versus time and are reported in table 1. It follows that PGA-analogues without NO₂ substituents are preferred.

TABLE 1
reaction rate of PGA-type molecules with urea

| Entry | Substituent | k_{PFO} (min⁻¹) |
|---|---|---|
| 1a | H | 0.085 ± 0.003 |
| 1b | p-Me | 0.061 ± 0.005 |
| 1c | p-NO₂ | 0.006 ± 0.002 |

### 2.2 General Method for Providing Monomers of General Formula (I)

Monomers can be purchased from commercial suppliers when available, or can be prepared by chemical synthesis. For this, acylation reactions are suitable, starting from styrene or isopropenylbenzene or other derivatives that are generally commercially available. Ethynylbenzene is another suitable starting material, from which a vinyl analogue can be obtained after acylation by partial hydrogenation of the triple bond.

### 2.3 Synthesis of p-(Vinylphenyl)Ethenone (VPE)

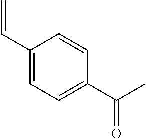

In a 3-neck round bottom flask p-(ethynylphenyl)ethenone (10.0 g, 69.4 mmol) was suspended in EtOH (350 mL) and Lindlar's catalyst (300 mg, 3 w %) was added. Air was replaced by H₂ and the suspension was stirred at RT for 2-16 hours. To monitor the conversion (and thus preventing over-reduction of VPE into the alkane), samples were frequently taken from the reaction mixture and, after evaporation of EtOH under reduced pressure, the conversion was determined by ¹H-NMR (CDCl₃). After the conversion was >90%, the H₂-filled balloon was removed and the reaction mixture was concentrated under reduced pressure. The crude product was re-dissolved in CH₂Cl₂ and purified by filtration over Hyflo. The filtrate was concentrated under reduced pressure, giving crude VPE as a yellow liquid in a 99% yield (10.1 g, 69.0 mmol). Melting point 29° C., melt enthalpy 90.6 J/g. ¹H-NMR (CDCl₃, 600 MHz) δ 7.92 (d, J=8.3 Hz, 2H), 7.48 (d, J=8.2 Hz, 2H), 6.75 (dd, J=17.6 Hz, 10.9 Hz, 1H), 5.87 (d, J=17.6 Hz, 1H), 5.39 (d, J=10.9 Hz, 1H), 2.59 (s, 3H).

### 2.4 Synthesis of 2-bromo-1-(4-ethenylphenyl)ethan-1-one

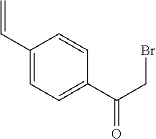

In a round bottom flask p-(ethynylphenyl)bromo-ethenone (15.5 g, 69.4 mmol) is suspended in EtOH (350 mL) and Lindlar's catalyst (300 mg, 3 w %) is added. Br Air is replaced by H₂ and the suspension is then stirred at RT for 2-16 hours. Further following the procedure described in example 2.3, the crude precursor 0 monomer can be obtained as a yellow/brownish liquid.

### 2.5 Synthesis of 2,2-dihydroxy-1-(4-ethenylphenyl)ethan-1-one

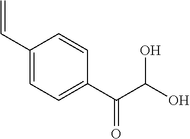

VPE (146 mg, 1.0 mmol, 1.0 eq.) is dissolved in a 10:1 mixture of dioxane (3 mL) and H₂O (0.3 mL) in a microwave tube equipped with a magnetic stirrer. Selenium dioxide (2.0 mmol, 2.0 eq.) is added and the tube sealed. The mixture is shaken vigorously until selenium dioxide is completely dissolved and the tube placed in the microwave in which it is heated for 5 minutes at 180° C. The crude reaction mixture is impregnated on silica and purified over silica (EtOAc:hexane) to give pure 2,2-dihydroxy-1-(4-ethenylphenyl)ethan-1-one.

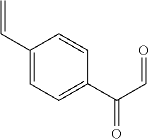

### 2.6 Synthesis of 1-(4-ethenylphenyl)ethan-1,2-dione

2,2-dihydroxy-1-(4-ethenylphenyl)ethan-1-one is dried under vacuum (>1 mbar) over anhydrous P₂O₅ for 24 hours at RT, resulting in 1-(4-ethenylphenyl)ethan-1,2-dione.

### 2.7 Synthesis of (4-ethenylphenyl)(2-dioxolanyl)methanone

2,2-dihydroxy-1-(4-ethenylphenyl)ethan-1-one (1 mmol, 1 eq.) is dissolved in ethylene glycol and 1 vol.-% AcOH (5 mL) and stirred at RT overnight. CH₂Cl₂ is then added and the mixture transferred to a separation funnel. The organic layer is washed with water three times to remove ethylene glycol and AcOH. Concentration of the organic layer then yields (4-ethenylphenyl)(2-dioxolanyl)methanone.

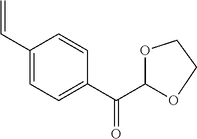

### Example 3—Polymerization

### General Method for Polymerization

Polymerization of monomers of general formula (I) can be performed using any known polymerization method, such as ionic polymerization (anionic, cationic), free radical polymerization, or controlled radical polymerization (RAFT, ATRP). Any sufficiently inert dissolving solvent can be used. Suspension polymerization is an attractive method because it can lead to granulated material. When a crosslinked sorbent is desired, up to 10% crosslinker can be added to the monomer mixture prior to polymerization, such as a divinylbenzene or butadiene. A skilled person can select suitable crosslinkers, which generally have more than one polymerizable moiety. Using about 0.5% to about 4% crosslinker gave good results. When a more hydrophilic sorbent is desired, hydrophilic comonomers can be added to the monomer mixture prior to polymerization, such as vinylbenzenesulfonic acid or acrylic acid. A skilled person can select suitable hydrophilic comonomers, which generally have a single polymerizable moiety and which also comprise a very polar group such as a carboxylic acid or a sulfonic acid. Because the polydispersity of the sorbent is not of high importance, it is efficient to let the polymerization run to completion, for example by letting it react overnight. This achieves high monomer economy and reduces the need for reaction monitoring. Purification can be done by precipitation in any solvent in which unreacted substances will dissolve, such as methanol. Alternately, the polymerization mixture can be used in the conversion as a crude mixture.

### 3.1 General Solution Polymerisation Method

Monomer (0.5 mmol) was dissolved in EtOH (2-10 mL) and divinylbenzene (1-4 eq.) and AIBN (1-3 mol %) were added. The flask was sealed and nitrogen was bubbled through the solution for 20 minutes. The solution was heated to 60° C. for 24 hours. The mixture was allowed to cool to room temperature and was centrifuged and the supernatant was removed. The resulting sorbent was washed and centrifuged with the EtOH 3 times and the last time washed with water. After centrifugation, the polymer was dried overnight over P₂O₅ under vacuum.

### 3.2 General Suspension Polymerisation Method

NaCl (10.5 mg), polyacrylic acid sodium salt (468 mg of a 10 w % gel in water), and Ca₃(PO₄)₂ (86 mg) were added to water (15 mL) in a glass reactor with mechanical stirrer, and stirred for 30 minutes. Monomer (15 mmol), porogen (2-3 mL of a non-water-miscible liquid), 80% divinylbenzene (1-6 mol %) and 50% benzoylperoxide blend with dicyclohexyl phthalate (1 mol %) were mixed separately and, after the initiator was dissolved, added to the aqueous phase. The mixture was stirred with a mechanical stirrer until an emulsion was obtained. Air was displaced by nitrogen in the glass reactor. The mixture was stirred at 73° C. for 16 hours, after which the suspension was filtered over a 200 μm filter. The resulting powder or beads in the residue were washed with acetone and water and dried over P₂O₅ under vacuum.

### 3.3 Preparation of Poly[(p-vinylphenyl)ethenone])-co-(divinylbenzene)]

The same procedure as for the preparation of polystyrene beads was employed, with some minor modifications. In brief, the aqueous phase was prepared by addition of NaCl (11 mg), polymethacrylic acid sodium salt solution (452 mg of a 10% gel in water) and CaHPO₄ (84 mg) to water (15 mL). The organic phase was composed of VPE (2.1 g, 14.4 mmol, 2 mL), porogen (2.9 mL, composition see table 2), 80% technical grade DVB (3-6 mol %) and a 50% benzoylperoxide blend with dicyclohexyl phthalate (174 mg, 0.36 mmol, 2.5 mol %). After mixing and polymerization (same procedure followed as for ‘Suspension Polymerization of Styrene’), the resulting suspension was allowed to cool to RT and poured over a filter (cut-off 200 μm, Veco B. V.). The residue was washed with acetone and water, and finally dried over P₂O₅ under vacuum, resulting in pVPE (1.1-1.9 grams, yield 52-90%).

TABLE 2
different p(VPE) polymers that were prepared

| | | Porogen | | Bead diameter | Yield | S_{BET} surface |
|---|---|---|---|---|---|---|
| # | Porogen | ratio | DVB | (mm) | (%) | area (m²/g) |
| 1^{A} | heptane/toluene | 75:25 | 3% | 0.62 ± 0.22 | 93% | <0.05 |
| 2 | heptane/toluene | 50:50 | 3% | 0.40 ± 0.28 | 99% | <0.05 |
| 3 | heptane/toluene | 40:60 | 3% | 0.48 ± 0.14 | 65% | 0.1 |
| 4 | heptane/toluene | 30:70 | 3% | 0.61 ± 0.23 | 75% | 2.0 (1.9^{b}) |
| 5 | heptane/toluene | 20:80 | 3% | 0.66 ± 0.21 | 66% | 0.2 |
| 6 | heptane/toluene | 10:90 | 3% | 0.47 ± 0.10 | 70% | <0.05 |
| 7 | toluene | — | 6% | 0.71 ± 0.23 | 69% | 0.2 |
| 8^{A} | toluene/ | 90:10 | 3% | 0.57 ± 0.34 | 52% | <0.05 |
| | nitrobenzene | | | | | |
| 9 | toluene/ | 80:20 | 6% | 0.55 ± 0.19 | 82% | <0.05 |
| | nitrobenzene | | | | | |

^{a}Aggregated particles were obtained, ^{b}Surface area after oxidation of pVPE.

### Example 4—Conversion of Polymerized Monomers

### General Methods for Conversion of Monomers of General Formula (I)

Table 3 shows suitable conversion methods for different monomers of general formula (I). Purification can be done by precipitation in any solvent in which unreacted substances will dissolve, such as methanol.

TABLE 3
suitable conversion methods for different monomers of general formula (1)

| Monomer type | Conversion method |
|---|---|
| 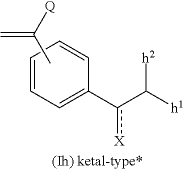 | when either or both of h¹ and h² are halogen, oxidation will lead to a PGA- type moiety. Oxidation using DMSO at elevated temperatures is convenient. when h² and h¹ together form an acetal, hemiacetal, or a thio- or amino-version thereof, hydrolysis, preferably under mild acidic conditions, leads to a PGA-type moiety. |
| 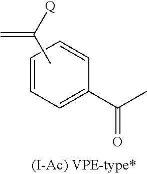 | i) halogenation (at acetyl), followed by ii) oxidation; The above is conveniently performed using hydrohalic acid in DMSO; direct oxidation at acetyl is also an option. |
| 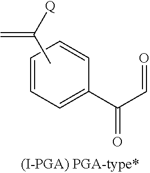 | None required |
| 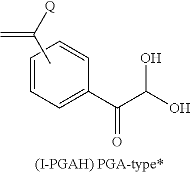 | None required |

*Q, X, h¹, and h² are as defined elsewhere

### 4.1 Preparation of PGA-Type Sorbent Based on Poly-VPE

The different mixtures obtained in example 3.3 were converted to PGA-type sorbents. Being VPE-type material prior to conversion, conversion was performed using halogenation and Kurnblum Oxidation of the pVPE. The same procedure as for the halogenation and Kurnblum oxidation of acetylated polystyrene beads was employed (see Example 1.6) for 12 hours, downscaled to 600 mg pVPE per batch. After washing, 606 mg of yellow beads (pVPE-Ox) was obtained.

The acetyl aromatic groups in pVPE beads were thus halogenated and subsequently converted into PGAH-groups by a Kornblum oxidation in a one-pot procedure. To establish the optimal reaction time for these oxidizing conditions to obtain the highest PGA/PGAH-density, beads were taken from the reaction mixture at different time points, and their urea binding capacity was determined (see example 4). Urea binding capacity of the beads increased with oxidation time during the first 8 hours to over 2 mmol/g, demonstrating successful oxidation of the acetyl group into PGAH/PGA. For comparison, oxidation of acetylated PS-beads (not using precursor monomers) could not achieve this binding capacity (see example 4).

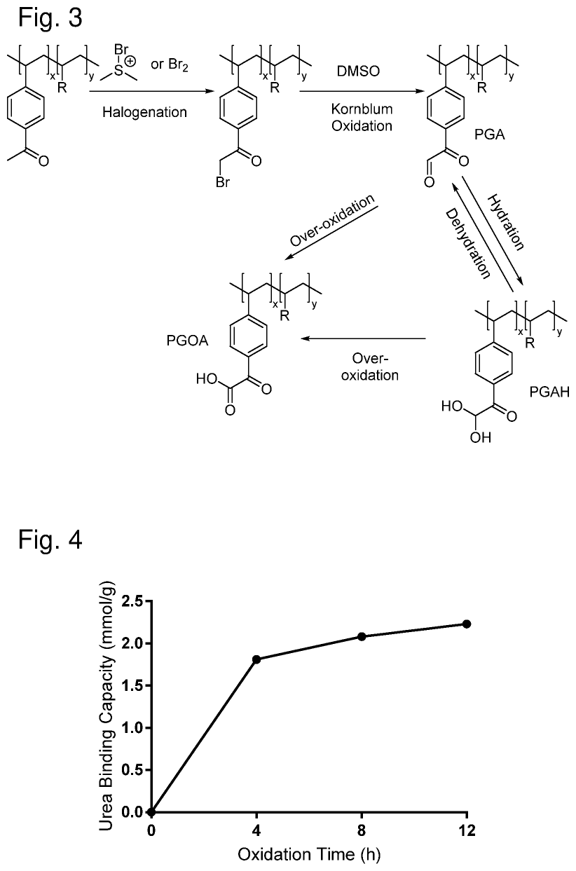

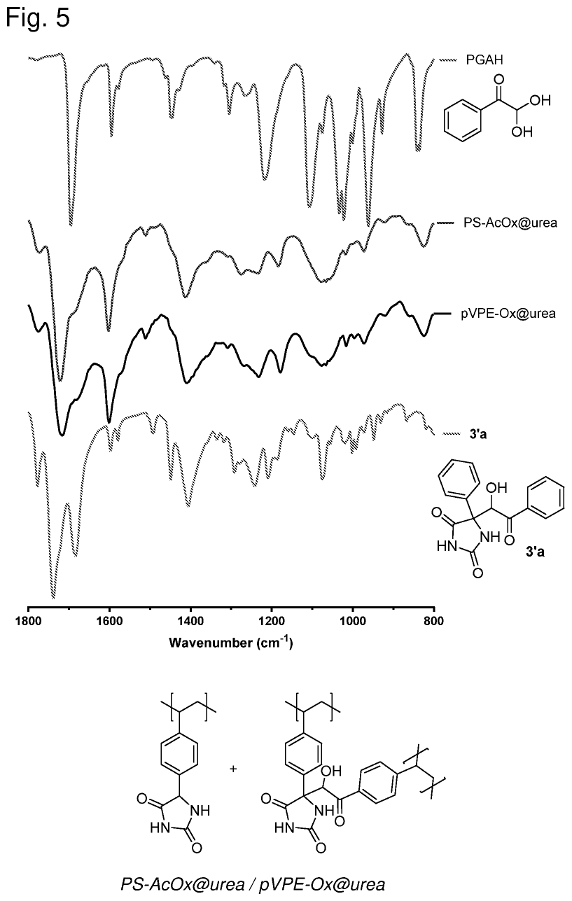

At longer reaction times, the binding capacity decreased. IR analysis (FIG. 5) of the sorbent obtained after 8 h of oxidation showed a single carbonyl peak at 1675 cm⁻¹ with a minor shoulder peak at 1740 cm⁻¹, whereas the sorbent obtained after 48 hours of oxidation showed two carbonyl peaks at 1675 cm⁻¹ and 1740 cm-1. This is likely due to over-oxidation (see FIG. 3). The shoulder peak at 1740 cm⁻¹ in the 8 h sample indicates that the over-oxidation of PGA/PGAH into PGOA already occurred during the first 8 hours, but it is slower than the oxidation of the acetyl group into the PGA/PGAH group. To avoid this over-oxidation reduced presence of HBr, or reduced reaction time is desirable. For oxidation, the reaction time is preferably at most 32 hours, more preferably at most 24 hours, even more preferably at most 16 hours, most preferably at most about 8 hours.

### 4.2 Preparation of PGA-Type Sorbent Based on Poly(VPE-Br)

Essentially, this material can be treated as poly-VPE without the need for halogenation. Polymer resin obtained via the general suspension polymerisation method described above (500 mg) is swollen in DMSO and stirred with a mechanical stirrer. Trimethylamine is used as a base for the DMSO-oxidation, which takes place once the suspension is heated to 80° C. for 8 hours. Washing is done as for example 1.6, after which PGA-type sorbent is obtained.

### 4.3 Preparation of PGA-Type Sorbent Based on Poly(Protected PGA)

Essentially this material, which is polymerized (4-ethenylphenyl) (2-dioxolanyl) methanone (see Example 2.7), is a protected PGA-type sorbent, i.e. a PGA-type sorbent wherein the glyoxal moiety is acetal-protected. Resin is swollen in THF, after which catalytic acetic acid and 5 vol.-% water is added to deprotect the glyoxal moiety. The solvents are filtered off two times, after which a new batch of THF, water, and acetic acid is added. Then the sorbent is washed with water twice, and dried as described in example 1.6, leading to a PGA-type solvent.

### Example 5—Analysis of Sorbents

### General Methods for Determining Urea Binding Capacity

Sorbent (10 mg or 15 mg) was suspended in urea-enriched PBS (30 mM, 1 mL or 1.5 mL) in a 1.5 mL microcentrifuge tube (Eppendorf, individual tube for each timepoint) and placed at 37° C. for a set amount of time. The sorbent was spun down in the tube (12.000 rpm in a conventional benchtop centrifuge, 5 min) and the urea concentration was determined in the supernatant using a commercially available urease assay (Urea CT*FS** colorimetric test purchased at DiaSys Diagnostic Systems GmbH, Holzheim, Germany). In brief, this test determines urea concentrations via a coupled enzyme reaction, which results in a colorimetric (570 nm) product in a concentration proportional to the urea concentration. To determine the maximum urea binding capacity a sample was placed at 70° C. for 24 hours and the urea concentration was determined in the supernatant. In an alternate method, sorbent (15 mg) was suspended in a solution of urea in PBS (1.5 mL, 30 mM or 50 mM) in a 1.5 mL Eppendorf and were placed in a rotation oven at 70° C. After 24 hours the sample was allowed to cool to RT and the urea concentration was determined in the supernatant by a standard urease assay (a urea stock solution kept for 24 hours at 70° C. was used as a negative control). The urea binding capacity of the sorbent was calculated based on the difference in urea concentration of the supernatant of the sorbent and the control solution.

### 5.1 Analysis of Beads Derived from Styrene

PS-based sorbents were analyzed by quantitative ¹³C-solid state NMR to quantify the amount of PGAH groups in Ps-Ac-Ox. The CH₃ peak of the acetyl group detected in ¹³C-NMR spectrum of PS-Ac had disappeared, indicating that all acetyl groups had been converted. Comparison of the area under the hydrate carbon peak (80-100 ppm) with that of the aromatic peaks (110-160 ppm) and the aliphatic peaks (10-50 ppm) showed that ˜40% of the aromatic groups (thus ˜67% of the acetyl groups) had been converted into PGAH groups. In addition, a minor peak around 165 ppm was detected, which is assigned to the carboxylic acid carbonyl peak from PGOA.

PS-Ac beads were oxidized for 8 hours on a 60 gram scale and the obtained beads (Ps-Ac-Ox) were characterized by SEM, light microscopy and nitrogen physisorption. PS—Ac-Ox showed similar size (0.54±0.11 mm), surface area (37.0 m²/g) and pore volume and pore size/volume distribution as PS and PS-Ac. This confirms that the oxidation reaction neither affects the macroporosity nor degrades the beads, likely because the reaction temperature (80° C.) was below the glass transition temperatures (T₉) of both the PS-Ac and PS—Ac-Ox beads (T₉ of dry beads were 184° C. and >230° C., respectively). The PGAH content of the sorbent according to ¹³C-NMR was similar to the sorbent prepared at small scale. The urea binding capacity of Ps-Ac-Ox was 1.4 mmol/g. Table 4 shows properties of these sorbents.

TABLE 4
properties of reference materials

| | | | | | | Urea |
|---|---|---|---|---|---|---|
| | | Surf. | Pore | | | Binding |
| | Diameter | Area | Vol. | | | Cap. |
| Beads | (mm) | (m²/g) | (mL/g) | Functionalization | | (mmol/g) |
| PS | 0.49 ± 0.18 | 36.3 | 0.32 | — | | — |
| PS-Ac | 0.61 ± 0.17 | 43.4 | 0.31 | ~60% | acetylation | — |
| Ps-Ac-Ox | 0.54 ± 0.11 | 37.0 | 0.31 | ~40% | PGAH | 1.4 |

### 5.2 Analysis of Beads Derived from a Precursor Monomer

Beads of table 2, entry 2 (S_{BET}<0.05 m²/g) and entry 4 (S_{BET}=2.0 m²/g) were selected for conversion. The beads of low surface area (entry 2) were oxidized for 4-12 hours under the same conditions as applied for PS-Ac. The urea binding capacities of the resulting oxidized pVPE beads (pVPE-Ox-(2) were 1.8-2.2 mmol/g, of which the highest binding capacity (2.2 mmol/g) was obtained after 12 hours of oxidation (see FIG. 4). The pVPE beads with the highest surface area (entry 4) were therefore oxidized for 12 hours and the urea binding capacity of these beads (pVPE-Ox-(4)) was 1.8 mmol/g. The surface area of the pVPE-Ox-(4) determined by nitrogen physisorption was similar to that of the corresponding pVPE beads (1.9 vs 2.0 m²/g), most likely because the reaction temperature of the oxidation reaction (80° C.) is much lower than the T₉ of pVPE beads (147° C.) and the beads therefore remain dimensionally stable under these oxidizing reaction conditions.

The VPE-based materials showed a higher urea binding capacity than the styrene-based materials which may be because of the increase in the density of acetyl groups and therefore a higher PGAH content after oxidation (1.4 vs. 1.8-2.2 mmol/g). Surprisingly, the surface area of pVPE beads had no influence on the urea binding capacity (1.8-2.2 and 1.8 mmol/g for pVPE-Ox-(2) and pVPE-Ox-(4) respectively). This shows that PGAH groups are accessible for urea also in materials without macroporosity, possibly because the sorbents swell to a minor but sufficient extent in water due to the polar and hydrophilic carbonyl groups and optionally the carboxylic acid groups of PGOA (FIG. 3). In addition, upon urea binding the beads become more hydrophilic further enhancing accessibility for water and urea thereby further improving urea binding kinetics. The average size of pVPE-Ox-(4) beads determined by light microscopy was slightly larger than that of the pVPE beads (0.77±0.20 and 0.61±0.23 mm, respectively). Due to the swelling/deswelling of the beads during nitrogen physisorption experiments, the pore/volume distribution for these materials was not determined. The pVPE-Ox-(4) and corresponding pVPE beads were analyzed by SEM and appeared to be hollow (deflated after drying under vacuum, which suggests core-shell phase separation during the polymerization reaction).

To determine the density of PGAH-groups in pVPE-Ox-(2) and pVPE-Ox-(4), these materials were analyzed by ¹³C solid state NMR spectroscopy. Comparison of the hydrate peak integral (80-100 ppm) with the backbone peak integral (10-50 ppm) demonstrates a PGAH-content of ˜50% for both pVPE-Ox-(2) and pVPE-Ox-(4), which confirms that higher PGAH contents are obtained using the VPE instead of the styrene route (˜50 and ˜40% respectively).

### 5.3 Analysis of Urea Binding Behaviour

It was found that the pVPE-Ox sorbent beads, which were for ˜50% functionalized with PGAH groups, had a urea binding capacity of ˜2 mmol/g. However, a 100% functionalized sorbent contains 5.5 mmol/g PGAH groups (including 3% crosslinker) based on molecular weight of the monomer (178 g/mol), which implies that a sorbent with ˜50% PGAH groups would have a urea binding capacity of 2.8 mmol/g at most. There are two reasons why the actual urea binding capacity for a sorbent functionalized with PGAH groups is lower than theoretical urea binding capacity based on a 1:1 reaction of urea with PGAH. First, some of the PGAH groups might be inaccessible for urea. Second, PGAH can react with urea in both a 1:1 and a 2:1 ratio (see FIG. 5 structure 3′a) and therefore one potential binding site is lost when PGAH reacts with urea in a 2:1 ratio. Quantification of the inaccessible and therefore unreacted PGAH groups in beads with ˜2 mmol urea per gram sorbent with ¹³C-NMR spectroscopy is not possible because unreacted PGAH and reacted PGAH give rise to signals in the same region of the spectrum. Therefore the sorbent beads which had reacted with urea (PS-AC-Ox@urea and pVPE-Ox-(4)@urea) were analyzed with IR spectroscopy, along with PGAH, and the 2:1 adduct of PGAH and urea (3′a) (FIG. 5). PGAH shows a clear ketone-carbonyl stretching vibration at 1700 cm-1 and a C—O stretching vibration at 1210 cm-1. However, these peaks have a lower intensity in the IR spectra of PS—Ac-Ox@urea and pVPE-Ox-(4)@urea, and the main carbonyl peak is clearly shifted (from 1700 to 1740 cm-1). Based on these observations it is concluded that the majority of the PGAH groups were indeed accessible for reaction with urea and had reacted. This agrees with the observation that the surface area does not influence the urea binding capacity. Moreover, the IR spectra of PS-AC-Ox@urea and pVPE-Ox-(4)@urea are more similar to the IR spectrum of the isolated 2:1 addition product 3′a. The several peaks arising from the carbonyl stretching vibration in the region of 1650-1800 cm-1 of 3′a are also present in the spectra of PS-AC-Ox@urea and pVPE-Ox-(4)@urea. Therefore it could be concluded that reaction of the 1:1 PGAH:urea adduct with a second PGA group takes place in the sorbent beads at least to some extent, explaining the difference between the urea binding capacity of the sorbents (˜2.0 mmol/g) and the theoretical capacity based on the actual PGAH content (2.8 mmol/g).

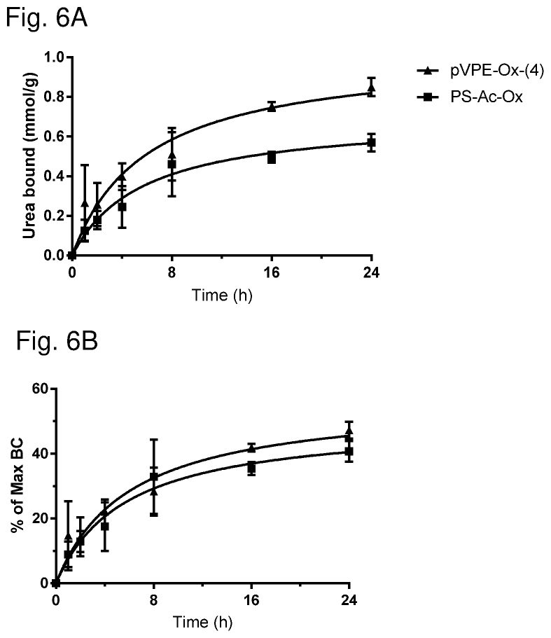

The kinetics of the urea binding of the two different types of PGA-type sorbents was investigated by incubating them in a 30 mM urea solution in phosphate buffered saline (PBS) at 37° C., conditions representative for the regeneration of dialysate. The urea binding was determined by measuring the urea concentration in the solution at different time points (FIG. 6A). The PS—Ac-Ox sorbent only showed a binding of 0.5-0.6 mmol/g after 24 hours. The sorbent pVPE-Ox-(4) had already bound 0.5-0.6 mmol per gram in 8 hours which increased to 0.8-0.9 mmol/g after 24 hours, reaching around 50% of its maximum binding capacity. FIG. 6B shows that both materials reached ˜45% of the maximum binding capacity after 24 hours.

### 5.4 Maximum Binding Capacity of Various Sorbents

Maximum urea binding capacities were determined for different sorbents, either PS-based or precursor-based. Table 5 shows urea binding capacities of sorbent beads in urea solution in PBS (10 mg/mL) at 70° C. for 24 hours under static conditions.

TABLE 5
urea binding for various PGA-type sorbents

| | [Urea]ₜ₌₀ (mM) | | [Urea]_{t=24 h} (mM) | | Max capacity |
|---|---|---|---|---|---|
| Sorbent | #1 | #2 | #1 | #2 | (mmol/g) |
| PS- Ac-Ox-1h | 30.5 | 30.4 | 28.1 | 27.3 | 0.28 ± 0.06 |
| PS-Ac-Ox-4h | 30.5 | 30.4 | 17.4 | 17.6 | 1.30 ± 0.01 |
| PS-Ac-Ox-8h (60 g) | 46.1 | 43.4 | 30.6 | 30.8 | 1.41 ± 0.01 |
| PS-Ac-Ox-24h | 30.5 | 30.4 | 16.2 | 16.6 | 1.41 ± 0.03 |
| PS-Ac-Ox-32h | 30.5 | 30.4 | 21.6 | 20.8 | 0.93 ± 0.06 |
| PS-Ac-Ox-48h | 30.5 | 30.4 | 27.0 | 27.5 | 0.32 ± 0.04 |
| pVPE-Ox-(2)-4h | 49.8 | 50.0 | 32.1 | 31.6 | 1.81 ± 0.04 |
| pVPE-Ox-(2)-8h | 49.8 | 50.0 | 29.0 | 29.2 | 2.08 ± 0.01 |
| pVPE-Ox-(2)-12h | 49.8 | 50.0 | 27.3 | 27.9 | 2.23 ± 0.04 |
| pVPE-Ox-(4) | 46.1 | 43.4 | 28.0 | 26.4 | 1.76 ± 0.11 |

### 5.5 Conclusion

PGA-type sorbent beads containing phenylglyoxaldehyde hydrate (PGAH) groups were successfully prepared via a precursor monomer using suspension polymerization followed by monomer conversion of the precursor into PGA (here: oxidation). The VPE route outperformed known routes using styrene, and also saves one post polymerization modification step, importantly resulting in a sorbent with higher PGAH content (˜50%, vs ˜40% for PS-based sorbents) and concomitantly higher binding capacity (over 1.8 mmol/g, vs 1.4 mmol/g for PS-based sorbents). The accessibility of the PGAH groups in the VPE-based sorbents is not dependent on the surface area of the material, possibly because the beads swell to a minor extent. The kinetics of urea sorption from simulated dialysate showed that ˜30% of the binding capacity is reached after 8 hours at 37° C. The best sorbent developed (pVPE-Ox-(4)) bound ˜0.5-0.6 mmol/g in 8 hours, which demonstrates that ˜700 grams of this PGAH-type sorbent is needed to remove the daily urea production of 400 mmol of end-stage kidney disease patients during a dialysis session of 8 hours.

### REFERENCES

Ashraf S. M. “A Laboratory Manual of Polymers” I. K. International Pvt Ltd, 8 Dec. 2008;
Becker and Russell, J. Org. Chem., 1963 DOI: 10.1021/jo01042a502;
Evenepoel P, Bammens B, Verbeke K, Vanrenterghem Y. Kidney Int 2006; 70:794-9;
De Fijter C W, Oe P L, Nauta J J, et al. Adv Perit Dial 1991; 7:186-9;
Gardner et al., Appl Biochem Biotechnol. 1984; 10:27-40;
Gotch F A. Kinetic modeling of continuous flow peritoneal dialysis. Semin Dial 2001; 14:378-83;
Katritzky et al., Chem. Rev. (2004) DOI: 10.1021/cr020750m;
Perl J, Wald R, Bargman J M, et al. Clin J Am Soc Nephrol 2012; 7:1145-54;
Piraino B, Sheth H. Blood Purif 2010; 29:145-9;
Marminon et al., (2015, DOI: 10.1016/j.tetlet.2015.02.086;
“Metal-Catalyzed Cross-Coupling Reactions” 2^{nd} ed. (2008) DOI: 10.1002/9783527619535;
Nesrallah G E et al., J Am Soc Nephrol 2012; 23:696-705;
Susantitaphong P et al., Am J Kidney Dis 2012; 59:689-99;
Ting G O, Kjellstrand C, Freitas T, Carrie B J, Zarghamee S. Am J Kidney Dis 2003; 42:1020-35;
EP121275A1
U.S. Pat. No. 4,897,200A
DE2305186A1
U.S. Pat. No. 3,933,753A
WO2004078797A1
U.S. Pat. No. 4,178,241A
WO2017116515A1
WO2011102807A1
WO2019110557
WO2016126596

1. Method for producing a phenylglyoxaldehyde (PGA)-type sorbent, comprising the steps of:
i) providing monomers of general formula (I): 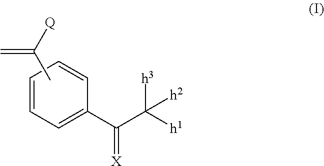
wherein:
Q is H or —CH₃;
h¹, h², and h³ are each independently chosen from H, halogen, —OH, —O(C₁₋₆ hydrocarbon), —S(C₁₋₆ hydrocarbon), —NH(C₁₋₆ hydrocarbon), or —N(C₁₋₆ hydrocarbon)₂; or h¹ and h² together form ═O; or h¹ and h² together form -o¹—(C₁₋₄ hydrocarbon)-o²- wherein of o¹ and o² are independently O, S, NH, or N(C₁₋₄ hydrocarbon); and
X is O, S, or NH;
ii) polymerizing the provided monomers to obtain a polymer; and
iii) converting polymerized monomers of general formula (I) that are not PGA-type monomers into PGA-type monomers.

2. The method according to claim 1, wherein the monomers of general formula (I) comprise monomers selected from:
1-(4-ethenylphenyl)ethan-1-one, 1-(3-ethenylphenyl)ethan-1-one, 1-(4-isopropenylphenyl)ethan-1-one, 1-(3-isopropenylphenyl)ethan-1-one,
2-bromo-1-(4-ethenylphenyl)ethan-1-one, 2-bromo-1-(3-ethenylphenyl)ethan-1-one, 2-bromo-1-(4-isopropenylphenyl)ethan-1-one, 2-bromo-1-(3-isopropenylphenyl)ethan-1-one,
2-chloro-1-(4-ethenylphenyl)ethan-1-one, 2-chloro-1-(3-ethenylphenyl)ethan-1-one, 2-chloro-1-(4-isopropenylphenyl)ethan-1-one, 2-chloro-1-(3-isopropenylphenyl)ethan-1-one,
1-(4-ethenylphenyl)ethan-1,2-dione, 1-(3-ethenylphenyl)ethan-1,2-dione, 1-(4-isopropenylphenyl)ethan-1,2-dione, 1-(3-isopropenylphenyl)ethan-1,2-dione,
2,2-dihydroxy-1-(4-ethenylphenyl)ethan-1-one, 2,2-dihydroxy-1-(3-ethenylphenyl)ethan-1-one, 2,2-dihydroxy-1-(4-isopropenylphenyl)ethan-1-one, and 2,2-dihydroxy-1-(3-isopropenylphenyl)ethan-1-one.

3. The method according to claim 1, wherein the monomer is of general formula (II-p): 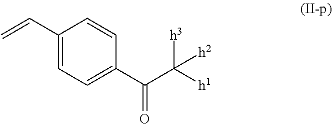
wherein h¹, h², and h³ are each independently chosen from H, halogen, —OH, —O(C₁₋₆ hydrocarbon), —S(C₁₋₆ hydrocarbon), —NH(C₁₋₆ hydrocarbon), or —N(C₁₋₆ hydrocarbon)₂; or h¹ and h² together form ═O; or h¹ and h² together form -o¹—(C₁₋₄ hydrocarbon)-o²- wherein of o¹ and o² are independently O, S, NH, or N(C₁₋₄ hydrocarbon).

4. The method according to claim 1, wherein comonomers are provided along with the monomers of general formula (I).

5. The method according to claim 1, wherein the polymer is crosslinked after polymerization or during polymerization.

6. The method according to claim 1, wherein conversion in step iii) comprises:
a) optional halogenation, preferably using halohydric acid; and
b) oxidation, preferably using dimethyl sulfoxide (DMSO).

7. The method according to claim 1, wherein in step iii) more than 30%, 35%, 40%, 45%, 50%, 55%, 60%, 65%, 70%, 75%, 80%, 85%, 90%, 95%, or 100% of the monomers of general formula (I) are converted into PGA-type monomers.

8. The method according to claim 1, wherein wherein:
Q is H; and/or
h¹ and h² are each independently chosen from H, halogen, —OH, and —O(C₁₋₄ hydrocarbon); or together form ═O; preferably h¹ and h² are both H, or are both —OH, or together form ═O; and/or
h³ is H; and/or
X is O.

9.-12. (canceled)

13. Method for removing nucleophilic waste solutes from a fluid, comprising the steps of:
i) providing a fluid comprising nucleophilic waste solutes, and
iia) contacting said fluid with a PGA-type sorbent as defined in claim 9, or alternately
iib) contacting said fluid with a dialysis fluid through a membrane, wherein the dialysis fluid is in contact with a PGA-type sorbent as defined in claim 9, and
iii) optionally, recovering the fluid.

14.-15. (canceled)

16. The method according to claim 4, wherein the comonomers are selected from the group consisting of styrene, isopropenylbenzene, divinylbenzene, vinylbenzenesulfonic acid, acrylic acid, methacrylic acid, methyl acrylate, methyl methacrylate, 2-hydroxyethyl 2-methylprop-2-enoate (HEMA), 2-hydroxypropyl 2-methylprop-2-enotate, 2-hydroxyethyl prop-2-enoate, 2-hydroxypropyl prop-2-enotate, N-(2-hydroxyethyl)methacrylamide, N-(2-hydroxypropyl)methacrylamide (HPMA), N-(2-hydroxyethyl)acrylamide, N-(2-hydroxypropyl)acrylamide, a telechelic N,N′-alkylenebisacrylamide such as N,N′-methylenebisacrylamide (NMAA), N-isopropylacrylamide (NIPAm), divinyl sulfone, butadiene, acrylonitrile, methacrylonitrile, vinylsulfonamide, N-alkyl vinylsulfonamide such as N-methyl vinylsulfonamide, and N,N-dialkyl vinylsulfonamide such as N,N-dimethyl vinylsulfonamide.
